(19) United States
(12) Patent Application Publication
(10) Pub. No.: US 2010/0087496 A1
(43) Pub. Date: Apr. 8, 2010

(54) NOVEL CINNAMIC AMIDE DERIVATIVES USEFUL AS ION CHANNEL MODULATORS

(75) Inventors: Antonio Nardi, Ballerup (DK); Morten Grunnet, Copenhagen O (DK); Joachim Demnitz, Copenhagen N (DK); Thomas Diness Jensen, Copenhagen (DK); Palle Christophersen, Ballerup (DK); David Spencer Jones, Smorum (DK); Elsebet Ostergaard Nielsen, Copenhagen K (DK); Dorte Strobaek, Farum (DK); Lars Siim Madsen, Soro (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/519,683

(22) PCT Filed: Dec. 17, 2007
(86) PCT No.: PCT/EP2007/064015

§ 371 Date: Jul. 24, 2009

Related U.S. Application Data
(60) Provisional application No. 60/870,781, filed on Dec. 19, 2006.

(30) Foreign Application Priority Data
Dec. 18, 2006 (DK) PA 2006 01657
Mar. 28, 2007 (DK) PA 2007 00481

(51) Int. Cl. A61K 31/165; C07C 233/01; C07D 257/04; A61K 31/41; A61P 15/00

(52) U.S. Cl. 514/381; 564/180; 548/252; 514/617

(57) ABSTRACT

This invention relates to novel cinnamic amide derivatives that are found to be potent modulators of ion channels and, as such, they are valuable candidates for the treatment of diseases or disorders as diverse as those which are responsive to modulation of ion channels.

### TECHNICAL FIELD

This invention relates to novel cinnamic amide derivatives that are found to be potent modulators of ion channels and, as such, they are valuable candidates for the treatment of diseases or disorders as diverse as those which are responsive to modulation of ion channels.

### BACKGROUND ART

Ion channels are cellular proteins that regulate the flow of ions through cellular membranes of all cells and are classified by their selective permeability to the different of ions (potassium, chloride, sodium etc.). Potassium channels, which represent the largest and most diverse sub-group of ion channels, selectively pass potassium ions and, doing so, they principally regulate the resting membrane potential of the cell and/or modulate their level of excitation. Chloride channels, as a further example, by selectively passing chloride channels are also important for setting cell resting membrane potential as well as they display a variety of other important physiological and cellular roles including regulation of pH, volume homeostasis, organic solute transport, cell migration, cell proliferation and differentiation.

Dysfunction of potassium channels and chloride channels, as well as other ion channels, generate loss of cellular control resulting in altered physiological functioning and disease conditions. Ion channel blockers and openers, by their ability to modulate ion channel function and/or regain ion channel activity in acquired or inherited channelopathies, are being used in the pharmacological treatment of a wide range of pathological diseases and have the potential to address an even wider variety of therapeutic indications. For instance, the primary indications for potassium channel openers encompass conditions as diverse as diabetes, arterial hypertension, cardiovascular diseases, urinary incontinence, atrial fibrillation, epilepsy, pain and cancer.

Among the large number of potassium channel types, the large-conductance calcium-activated potassium channel subtype is an obvious site for pharmacological intervention and one of the most exciting targets for the development of new potassium channel modulators. Their physiological role has been especially studied in the nervous system where they are key regulators of neuronal excitability and of neurotransmitter release, and in smooth muscle where they are crucial in modulating the tone of vascular, broncho-tracheal, urethral, uterine or gastro-intestinal musculature.

Given these implications, small agents with BK-opener properties, named BK-openers or BK-activators, could have a potentially powerful influence in the modulation and control of numerous consequences of muscular and neuronal hyperexcitability, such as asthma, urinary incontinence and bladder spasm, gastroenteric hypermotility, psychoses, post-stroke neuroprotection, convulsions and anxiety. As far as the cardiovascular system is concerned, the physiological function of these ion channels represents a fundamental steady state mechanism, modulating vessel depolarisation, vasoconstriction and increases of intravascular pressure.

In view of the ample documentation involving a dysfunction in the role and/or the expression of vascular BK channels in a number of cardiovascular diseases, the development of selective activators of BK channels is seen as a promising research field for the pharmacotherapy of vascular diseases, including hypertension, erectile dysfunction, coronary diseases and vascular complications associated with diabetes or hypercholesterolemia.

WO 99/07669, U.S. Pat. No. 6,046,239 and U.S. Pat. No. 6,127,392 describe anthranilic acid analogs useful as potassium channel and chloride channel modulators. However, the cinnamic amide derivatives of the present invention are not disclosed.

### SUMMARY OF THE INVENTION

Is an object of this invention to provide novel cinnamic amide derivatives useful as ion channel modulators. The cinnamic amide derivatives of the invention may be characterised by Formula I

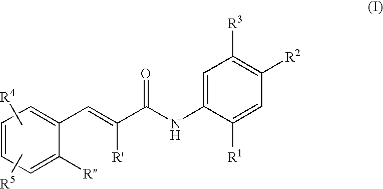

an enantiomer thereof or a mixtures of its enantiomers, or pharmaceutically-acceptable addition salts thereof, wherein

R¹ represents a substituent selected from the group consisting of nitro, amino, hydroxy, carboxy, sulfonic acid, sulfonic acid alkyl ester, sulfamoyl, acetamido, methyl-sulfonyl-amino, phenyl-sulfonyl-amino, N-methyl-sulfonyl-carboxamide (methyl-sulfonyl-amino-carbonyl), N-phenyl-sulfonyl-carboxamide (phenyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl, tetrazolyl-methoxy, 5-oxo-4,5-dihydro-[1,2,4]oxadiazol-3-yl and N-cyano-carboxamide;

R² and R³, independently of each other, represent hydrogen, halo, trifluoromethyl, hydroxy or phenyl, which phenyl may optionally be substituted with halo and/or trifluoromethyl;

R⁴ and R⁵, independently of each other, represent hydrogen, halo, trifluoromethyl, nitro and/or phenyl; or

R⁴ and R⁵ together with the aromatic ring to which they are attached form a benzo-fused carbocyclic aromatic ring; and

R′ and R″ represent hydrogen, or, together with the carbon atoms of the aromatic ring to which they are attached, form a bicyclic carbocyclic or heterocyclic ring selected from indolyl and 2H-chromenyl, which 2H-chromenyl may optionally be substituted with oxo to form a 2-oxo-2H-chromenyl derivative.

In another aspect the invention provides pharmaceutical compositions comprising a therapeutically effective amount of the cinnamic amide derivative of the invention.

In a third aspect the invention relates to the use of the cinnamic amide derivative of the invention for the manufacture of pharmaceutical compositions.

In a fourth aspect the invention provides a kit of parts comprising at least two separate unit dosage forms (A) and (B1) or (B2): (A) a cinnamic amide derivative according to the invention; and (B1) a phosphodiesterase inhibitor, or (B2) an agent that potentiates endothelium-derived hyperpolarizing factor-mediated responses; and optionally (C) instructions for the simultaneous, sequential or separate administration of the cinnamic amide derivative of A, and the phosphodiesterase inhibitor of B1, or an agent that potentiates endothelium-derived hyperpolarizing factor-mediated responses of B2, to a patient in need thereof.

In a further aspect the invention provides a method of treatment, prevention or alleviation of a disease or a disorder or a condition of a living animal body, including a human, which disorder, disease or condition is responsive to modulation of ion channels, which method comprises the step of administering to such a living animal body in need thereof, a therapeutically effective amount of the cinnamic amide derivative of the invention.

Other objects of the invention will be apparent to the person skilled in the art from the following detailed description and examples.

### DETAILED DISCLOSURE OF THE INVENTION

In its first aspect the invention provides novel cinnamic amide derivatives of Formula I

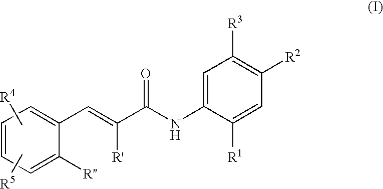

an enantiomer thereof or a mixtures of its enantiomers, or pharmaceutically-acceptable addition salts thereof, wherein

R¹ represents a substituent selected from the group consisting of nitro, amino, hydroxy, carboxy, sulfonic acid, sulfonic acid alkyl ester, sulfamoyl, acetamido, methyl-sulfonyl-amino, phenyl-sulfonyl-amino, N-methyl-sulfonyl-carboxamide (methyl-sulfonyl-amino-carbonyl), N-phenyl-sulfonyl-carboxamide (phenyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl, tetrazolyl-methoxy, 5-oxo-4,5-dihydro-[1,2,4]oxadiazol-3-yl and N-cyano-carboxamide;

R² and R³, independently of each other, represent hydrogen, halo, trifluoromethyl, hydroxy or phenyl, which phenyl may optionally be substituted with halo and/or trifluoromethyl;

R⁴ and R⁵, independently of each other, represent hydrogen, halo, trifluoromethyl, nitro and/or phenyl; or

R⁴ and R⁵ together with the aromatic ring to which they are attached form a benzo-fused carbocyclic aromatic ring; and

R′ and R″ represent hydrogen, or, together with the carbon atoms of the aromatic ring to which they are attached, form a bicyclic carbocyclic or heterocyclic ring selected from indolyl and 2H-chromenyl, which 2H-chromenyl may optionally be substituted with oxo to form a 2-oxo-2H-chromenyl derivative.

In a more preferred embodiment the cinnamic amide derivative of the invention is a compound of Formula II,

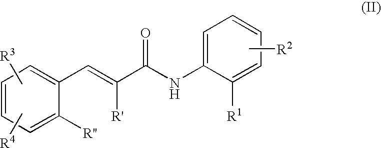

an enantiomer or a mixture of its enantiomers, or a pharmaceutically-acceptable addition salt thereof, wherein

R¹ represents a substituent selected from the group consisting of nitro, amino, hydroxy, carboxy, sulfonic acid, sulfamoyl, acetamido, methyl-sulfonyl-amino, N-methyl-sulfonyl-carboxamide (methyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl, tetrazolyl-methoxy, N-phenyl-sulfonyl-carboxamide (phenyl-sulfonyl-amino-carbonyl) and N-cyano-carboxamide;

R² represents hydrogen, halo (in particular chloro, bromo or iodo), trifluoromethyl, hydroxy or phenyl, which phenyl may optionally be substituted with halo (in particular fluoro or chloro) and/or trifluoromethyl;

R³ and R⁴, independently of each other, represent hydrogen, halo (in particular fluoro, chloro or bromo), trifluoromethyl, nitro and/or phenyl; or

R³ and R⁴ together with the aromatic ring to which they are attached form a benzo-fused carbocyclic aromatic ring (in particular naphthyl); and

R′ and R″ represent hydrogen, or, together with the carbon atoms of the aromatic ring to which they are attached, form a bicyclic carbocyclic or heterocyclic ring selected from indolyl and 2H-chromenyl, which 2H-chromenyl may optionally be substituted with oxo to form a 2-oxo-2H-chromenyl derivative.

In a preferred embodiment the cinnamic amide derivative of the invention is a compound of Formula I or II, wherein R¹ represents a substituent selected from the group consisting of nitro, amino, hydroxy, carboxy, sulfonic acid, sulfonic acid alkyl ester, sulfamoyl, acetamido, methyl-sulfonyl-amino, phenyl-sulfonyl-amino, N-methyl-sulfonyl-carboxamide (methyl-sulfonyl-amino-carbonyl), N-phenyl-sulfonyl-carboxamide (phenyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl, tetrazolyl-methoxy, 5-oxo-4,5-dihydro-[1,2,4]oxadiazol-3-yl and N-cyano-carboxamide.

In a more preferred embodiment R¹ represents a substituent selected from the group consisting of nitro, amino, hydroxy, carboxy, sulfonic acid, sulfamoyl, acetamido, methyl-sulfonyl-amino, N-methyl-sulfonyl-carboxamide (methyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl, tetrazolyl-methoxy, N-phenyl-sulfonyl-carboxamide (phenyl-sulfonyl-amino-carbonyl) and N-cyano-carboxamide.

In another more preferred embodiment R¹ represents a substituent selected from the group consisting of carboxy, sulfonic acid, sulfonic acid alkyl ester, hydroxy, tetrazolyl and 5-oxo-4,5-dihydro-[1,2,4]oxadiazol-3-yl.

In a third more preferred embodiment R¹ represents a substituent selected from the group consisting of carboxy, sulfonic acid, hydroxy and tetrazolyl.

In a fourth more preferred embodiment R¹ represents carboxy.

In a fifth more preferred embodiment R¹ represents sulfonic acid.

In a sixth more preferred embodiment R¹ represents sulfonic acid alkyl ester.

In a seventh more preferred embodiment R¹ represents hydroxyl.

In an eight more preferred embodiment R¹ represents tetrazolyl.

In a ninth more preferred embodiment R¹ represents 1H-tetrazol-5-yl or 2H-tetrazol-5-yl.

In a tenth more preferred embodiment R¹ represents 5-oxo-4,5-dihydro-[1,2,4]oxadiazol-3-yl.

In another preferred embodiment the cinnamic amide derivative of the invention is a compound of Formula I or II, wherein R¹ represents a substituent selected from the group consisting of nitro, amino, sulfamoyl, acetamido, methyl-sulfonyl-amino, N-methyl-sulfonyl-carboxamide (methyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl-methoxy, 5-oxo-4,5-dihydro-[1,2,4]oxadiazol-3-yl, phenyl-sulfonyl-amino, N-phenyl-sulfonyl-carboxamide (phenyl-sulfonyl-amino-carbonyl) and N-cyano-carboxamide.

In a more preferred embodiment R¹ represents a substituent selected from the group consisting of nitro, amino, sulfamoyl, acetamido, methyl-sulfonyl-amino, N-methyl-sulfonyl-carboxamide (methyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl-methoxy, N-phenyl-sulfonyl-carboxamide (phenyl-sulfonyl-amino-carbonyl) and N-cyano-carboxamide.

In another more preferred embodiment R¹ represents a substituent selected from the group consisting of nitro, amino, hydroxy and carboxy.

In a third more preferred embodiment R¹ represents nitro.

In a fourth more preferred embodiment R¹ represents amino.

In a fifth more preferred embodiment R¹ represents a substituent selected from the group consisting of sulfonic acid, sulfamoyl, CH₃CONH, methyl-sulfonyl-amino and methyl-sulfonyl-amino-carbonyl.

In a sixth more preferred embodiment R¹ represents sulfonic acid.

In a seventh more preferred embodiment R¹ represents sulfamoyl.

In an eight more preferred embodiment R¹ represents CH₃CONH.

In a ninth more preferred embodiment R¹ represents methyl-sulfonyl-amino.

In a tenth more preferred embodiment R¹ represents methyl-sulfonyl-amino-carbonyl.

In an eleventh more preferred embodiment R¹ represents a substituent selected from the group consisting of trifluoromethyl-sulfonyl-amino, trifluoromethyl-acetyl-amino, 2,2,2-trifluoro-1-hydroxy-1-trifluoromethyl-ethyl, tetrazolyl, tetrazolyl-methoxy, 5-oxo-4,5-dihydro-[1,2,4]oxadiazol-3-yl, phenyl-sulfonyl-amino, N-phenyl-sulfonyl-carboxamide (phenyl-sulfonyl-amino-carbonyl) and N-cyano-carboxamide.

In a twelfth more preferred embodiment R¹ represents a substituent selected from the group consisting of trifluoromethyl-sulfonyl-amino, trifluoromethyl-acetyl-amino, 2,2,2-trifluoro-1-hydroxy-1-trifluoromethyl-ethyl, tetrazolyl, tetrazolyl-methoxy, N-phenyl-sulfonyl-carboxamide (phenyl-sulfonyl-amino-carbonyl) and N-cyano-carboxamide.

In a thirteenth more preferred embodiment R¹ represents trifluoromethyl-acetyl-amino.

In a fourteenth more preferred embodiment R¹ represents 2,2,2-trifluoro-1-hydroxy-1-trifluoromethyl-ethyl.

In a fifteenth more preferred embodiment R¹ represents tetrazolyl.

In a sixteenth more preferred embodiment R¹ represents tetrazolyl-methoxy.

In a seventeenth more preferred embodiment R¹ represents 5-oxo-4,5-dihydro-[1,2,4]oxadiazol-3-yl.

In an eighteenth more preferred embodiment R¹ represents phenyl-sulfonyl-amino.

In a nineteenth more preferred embodiment R¹ represents N-phenyl-sulfonyl-carboxamide (phenyl-sulfonyl-amino-carbonyl).

In a twentieth more preferred embodiment R¹ represents N-cyano-carboxamide.

In a third preferred embodiment the cinnamic amide derivative of the invention is a compound of Formula I or II, wherein R² and R³, independently of each other, represent hydrogen, halo, trifluoromethyl, hydroxy or phenyl, which phenyl may optionally be substituted with halo and/or trifluoromethyl.

In a more preferred embodiment R² represents hydrogen; and R³ represents hydrogen, halo or trifluoromethyl.

In another more preferred embodiment R² represents hydrogen; and R³ represents hydrogen or halo, and in particular chloro.

In a third more preferred embodiment R² and R³ both represent hydrogen.

In a fourth more preferred embodiment R³ represents hydrogen; and R² represents hydrogen, halo, trifluoromethyl, hydroxy or phenyl, which phenyl may optionally be substituted with halo and/or trifluoromethyl.

In a fifth more preferred embodiment R³ represents hydrogen; and R² represents hydrogen, halo, trifluoromethyl or phenyl, which phenyl may optionally be substituted with halo and/or trifluoromethyl.

In a sixth more preferred embodiment R³ represents hydrogen; and R² represents hydrogen, halo or phenyl, which phenyl may optionally be substituted with halo and/or trifluoromethyl.

In a seventh more preferred embodiment R³ represents hydrogen; and R² represents halo or phenyl, which phenyl may optionally be substituted with halo and/or trifluoromethyl.

In an eighth more preferred embodiment R³ represents hydrogen; and R² represents hydrogen, halo or phenyl, which phenyl is substituted with halo and/or trifluoromethyl.

In a ninth more preferred embodiment R³ represents hydrogen; and R² represents halo or phenyl, which phenyl is substituted with halo and/or trifluoromethyl.

In a tenth more preferred embodiment R³ represents hydrogen; and R² represents halo or trifluoromethyl.

In an eleventh more preferred embodiment R³ represents hydrogen; and R² represents phenyl, which phenyl is optionally substituted with halo and/or trifluoromethyl.

In a twelfth more preferred embodiment R³ represents hydrogen; and R² represents phenyl, which phenyl is substituted with halo, and in particular fluoro or chloro.

In a thirteenth more preferred embodiment the cinnamic amide derivative of the invention is a compound of Formula I or II, wherein R² represents hydrogen, halo, trifluoromethyl, hydroxy or phenyl, which phenyl may optionally be substituted with halo and/or trifluoromethyl.

In a fourteenth more preferred embodiment the cinnamic amide derivative of the invention is a compound of Formula I or II, wherein R² represents hydrogen, halo, trifluoromethyl or hydroxyl.

In a fifteenth more preferred embodiment the cinnamic amide derivative of the invention is a compound of Formula I or II, wherein R² represents hydrogen, chloro, bromo, iodo, trifluoromethyl or hydroxyl.

In a sixteenth more preferred embodiment the cinnamic amide derivative of the invention is a compound of Formula I or II, wherein R² represents hydrogen.

In a seventeenth more preferred embodiment the cinnamic amide derivative of the invention is a compound of Formula I or II, wherein R² represents phenyl, which phenyl may optionally be substituted with halo, and in particular fluoro or chloro, and/or trifluoromethyl.

In an eighteenth more preferred embodiment the cinnamic amide derivative of the invention is a compound of Formula I or II, wherein R² represents phenyl, which phenyl may optionally be substituted with fluoro or chloro.

In a fourth preferred embodiment the cinnamic amide derivative of the invention is a compound of Formula I or II, wherein R⁴ and R⁵, independently of each other, represent hydrogen, halo, trifluoromethyl, nitro and/or phenyl; or R⁴ and R⁵ together with the aromatic ring to which they are attached form a benzo-fused carbocyclic aromatic ring (naphthyl).

In a more preferred embodiment R⁴ represents hydrogen; and R⁵ represents halo, trifluoromethyl, nitro or phenyl.

In another more preferred embodiment R⁴ represents hydrogen; and R⁵ represents chloro, bromo, trifluoromethyl, nitro or phenyl.

In a third more preferred embodiment R⁴ represents halo, and in particular fluoro or chloro, or trifluoromethyl; and R⁵ represents halo, and in particular chloro, or trifluoromethyl.

In a fourth more preferred embodiment R⁴ represents fluoro, chloro or trifluoromethyl; and R⁵ represents chloro or trifluoromethyl.

In a fifth more preferred embodiment R⁴ and R⁵ both represent hydrogen.

In a sixth more preferred embodiment R³ and R⁴, independently of each other, represent hydrogen, halo, trifluoromethyl, nitro and/or phenyl.

In a seventh more preferred embodiment R³ represents hydrogen; and R⁴ represents halo, trifluoromethyl, nitro or phenyl.

In an eighth more preferred embodiment R³ represents hydrogen; and R⁴ represents chloro, bromo, trifluoromethyl, nitro or phenyl.

In a ninth more preferred embodiment R³ represents halo, and in particular fluoro or chloro, or trifluoromethyl; and R⁴ represents halo, and in particular chloro, or trifluoromethyl.

In a tenth more preferred embodiment R³ represents fluoro, chloro or trifluoromethyl; and R⁴ represents chloro or trifluoromethyl.

In an eleventh more preferred embodiment R³ and R⁴ both represent hydrogen.

In a fifth preferred embodiment the cinnamic amide derivative of the invention is a compound of Formula I or II, wherein R⁴ and R⁵ together with the aromatic ring to which they are attached form a benzo-fused carbocyclic aromatic ring (naphthyl).

In a more preferred embodiment R³ and R⁴ together with the aromatic ring to which they are attached form a benzo-fused carbocyclic aromatic ring (naphthyl).

In a sixth preferred embodiment the cinnamic amide derivative of the invention is a compound of Formula I or II, wherein R′ and R″ represent hydrogen, or, together with the carbon atoms of the aromatic ring to which they are attached, form a bicyclic carbocyclic or heterocyclic ring selected from indolyl and 2H-chromenyl, which 2H-chromenyl may optionally be substituted with oxo to form a 2-oxo-2H-chromenyl derivative.

In a more preferred embodiment R′ and R″ both represent hydrogen.

In another more preferred embodiment R′ and R″, together with the carbon atoms of the aromatic ring to which they are attached, form a bicyclic carbocyclic or heterocyclic ring selected from indolyl and 2H-chromenyl, which 2H-chromenyl may optionally be substituted with oxo to form a 2-oxo-2H-chromenyl derivative.

In a seventh preferred embodiment the cinnamic amide derivative of the invention is a compound of Formula I or II, wherein R¹ represents tetrazolyl; R² represents hydrogen, halo, 4-fluoro-phenyl, 4-chloro-phenyl; and R³ represents hydrogen or halo.

In a more preferred embodiment R⁴ represents hydrogen; and R⁵ represents halo, trifluoromethyl, nitro or phenyl; or R⁴ represents halo, and in particular fluoro or chloro, or trifluoromethyl; and R⁵ represents halo, and in particular chloro, or trifluoromethyl.

In another more preferred embodiment R⁴ represents halo, and in particular fluoro or chloro; and R⁵ represents halo, and in particular chloro, or trifluoromethyl.

In a third more preferred embodiment R⁴ represents halo, and in particular fluoro; and R⁵ represents trifluoromethyl.

In a fourth more preferred embodiment R⁴ and R⁵ both represent halo, and in particular chloro.

In a fifth more preferred embodiment R⁴ and R⁵ both represent trifluoromethyl.

In a sixth more preferred embodiment

R¹ represents tetrazolyl;

R² represents halo, 4-fluoro-phenyl, 4-chloro-phenyl;

R³ represents hydrogen; and R⁴ represents trifluoromethyl; or

R³ represents halo or trifluoromethyl; and R⁴ represents halo, and in particular chloro, or trifluoromethyl; and R′ and R″ both represent hydrogen.

In an eighth preferred embodiment the cinnamic amide derivative of the invention is a compound of Formula I or II, wherein

R¹ represents tetrazolyl;

R² represents hydrogen, halo, and in particular bromo, or 4-fluoro-phenyl;

R³ represents hydrogen or halo, and in particular chloro;

R⁴ represents hydrogen;

R⁵ represents halo, and in particular chloro or bromo; and

R′ and R″, together with the carton atoms to which they are attached and with the aromatic ring, form a bicyclic carbocyclic or heterocyclic ring selected from indolyl and 2H-chromenyl, which 2H-chromenyl may optionally be substituted with oxo to form a 2-oxo-2H-chromenyl derivative.

In a more preferred embodiment the cinnamic amide derivative of the invention is a compound of Formula I or II, wherein

R¹ represents tetrazolyl;

R² represents halo, and in particular chloro or bromo, 4-fluoro-phenyl;

R³ represents hydrogen;

R⁴ represents halo, and in particular chloro or bromo; and

R′ and R″, together with the carton atoms to which they are attached and with the aromatic ring, form a bicyclic carbocyclic or heterocyclic ring selected from indolyl and 2H-chromenyl, which 2H-chromenyl may optionally be substituted with oxo to form a 2-oxo-2H-chromenyl derivative.

In a ninth preferred embodiment the cinnamic amide derivative of the invention is a compound of Formula I or II, wherein

R¹ represents tetrazolyl;

R² represents hydrogen or halo, and in particular bromo;

R³ represents hydrogen or halo, and in particular chloro;

R⁴ and R⁵ together with the aromatic ring to which they are attached form a benzo-fused carbocyclic aromatic ring (naphthyl); and

R′ and R″ both represent hydrogen.

In a more preferred embodiment the cinnamic amide derivative of the invention is a compound of Formula I or II, wherein

R¹ represents tetrazolyl;

R² represents halo, and in particular chloro or bromo;

R³ and R⁴ together with the aromatic ring to which they are attached form a benzo-fused carbocyclic aromatic ring (naphthyl); and

R′ and R″ both represent hydrogen.

In a most preferred embodiment the cinnamic amide derivative of the invention is
6-Chloro-2H-chromene-3-carboxylic acid [5-chloro-2-(1H-tetrazol-5-yl) -phenyl]-amide;
(E)-N-(5-Chloro-2-hydroxy-phenyl)-3-(3-nitro-phenyl)-acrylamide;
(E)-N-[5-Chloro-2-(1H-tetrazol-5-yl)-phenyl]-3-(4-fluoro-3-trifluoromethyl-phenyl)-acrylamide;
(E)-3-(3,5-Bis-trifluoromethyl-phenyl)-N-(5-chloro-2-hydroxy-phenyl)-acrylamide;
(E)-N-[4-Bromo-2-(2H-tetrazol-5-yl)-phenyl]-3-(3-trifluoromethyl-phenyl)-acrylamide;
5-Chloro-1H-indole-2-carboxylic acid [4′-fluoro-3-(1H-tetrazol-5-yl)-biphenyl-4-yl]-amide;
(E)-3-(3,5-Bis-trifluoromethyl-phenyl)-N-[4′-chloro-3-(2H-tetrazol-5-yl)-biphenyl-4-yl]-acrylamide;
(E)-3-(3,5-Bis-trifluoromethyl-phenyl)-N-[4-iodo-2-(1H-tetrazol-5-yl)-phenyl]-acrylamide;
(E)-3-(3,5-Bis-trifluoromethyl-phenyl)-N-[4-bromo-2-(1H-tetrazol-5-yl)-phenyl]-acrylamide;
(E)-N-[4-Bromo-2-(1H-tetrazol-5-yl)-phenyl]-3-naphthalen-2-yl-acrylamide;
(E)-N-[5-Chloro-2-(1H-tetrazol-5-yl)-phenyl]-3-naphthalen-2-yl-acrylamide;
6-Chloro-2H-chromene-3-carboxylic acid [4-bromo-2-(1H-tetrazol-5-yl)-phenyl]-amide;
5-Chloro-1H-indole-2-carboxylic acid [5-chloro-2-(1H-tetrazol-5-yl)-phenyl]-amide;
(E)-N-[5-Chloro-2-(1H-tetrazol-5-yl)-phenyl]-3-(3,4-dichloro-phenyl)-acrylamide;
4-Chloro-2-((E)-3-naphthalen-2-yl-acryloylamino)-benzoic acid;
(E)-3-Naphthalen-2-yl-N-[2-(2,2,2-trifluoro-1-hydroxy-1-trifluoromethyl-ethyl)-phenyl]-acrylamide;
4-Chloro-2-[(E)-3-(3,4-dichloro-phenyl)-acryloylamino]-benzoic acid;
(E)-N-(2-Acetylamino-5-chloro-phenyl)-3-naphthalen-2-yl-acrylamide;
(E)-N-(5-Chloro-2-nitro-phenyl)-3-naphthalen-2-yl-acrylamide;
4-Chloro-2-((E)-3-naphthalen-2-yl-acryloylamino)-benzenesulfonic acid;
(E)-N-(5-Chloro-2-methanesulfonylamino-phenyl)-3-naphthalen-2-yl-acrylamide;
(E)-N-[5-Chloro-2-(2,2,2-trifluoro-acetylamino)-phenyl]-3-naphthalen-2-yl-acrylamide;
(E)-N-(2-Amino-5-chloro-phenyl)-3-naphthalen-2-yl-acrylamide;
(E)-N-(5-Chloro-4-fluoro-2-sulfamoyl-phenyl)-3-naphthalen-2-yl-acrylamide;
2-((E)-3-Biphenyl-4-yl-acryloylamino)-4-chloro-benzenesulfonic acid;
(E)-N-(5-Chloro-2-trifluoromethanesulfonylamino-phenyl)-3-naphthalen-2-yl-acrylamide;
(E)-N-[5-Chloro-2-(1H-tetrazol-5-ylmethoxy)-phenyl]-3-naphthalen-2-yl-acrylamide;
(E)-N-(2-Benzenesulfonylaminocarbonyl-5-chloro-phenyl)-3-naphthalen-2-yl-acrylamide;
6-Bromo-2-oxo-2H-chromene-3-carboxylic acid [5-chloro-2-(1H-tetrazol-5-yl)-phenyl]-amide;
(E)-N-(5-Chloro-2-methanesulfonylaminocarbonyl-phenyl)-3-naphthalen-2-yl-acrylamide;
4,5-Dichloro-2-((E)-3-naphthalen-2-yl-acryloylamino)-benzenesulfonic acid;
(E)-N-(2-Benzenesulfonylamino-5-chloro-phenyl)-3-naphthalen-2-yl-acrylamide;
4-Chloro-2-((E)-3-naphthalen-2-yl-acryloylamino)-benzoylcyanamide;
4-Chloro-2-((E)-3-naphthalen-2-yl-acryloylamino)-benzenesulfonic acid methyl ester;
(E)-N-[5-Chloro-2-(5-oxo-4,5-dihydro-[1,2,4]oxadiazol-3-yl)-phenyl]-3-naphthalen-2-yl-acrylamide; or
(E)-3-Naphthalen-2-yl-N-[2-(1H-tetrazol-5-yl)-phenyl]-acrylamide;

or a pharmaceutically-acceptable addition salt thereof.

Any combination of two or more of the embodiments described herein is considered within the scope of the present invention.

### DEFINITION OF SUBSTITUENTS

In the context of this invention halo represents fluoro, chloro, bromo or iodo.

In the context of this invention benzo-fused carbocyclic aromatic rings represents include naphthyl groups.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the lithium, and the ammonium salt, and the like, of a chemical compound of the invention containing an anionic group. Such cationic salts may be formed by procedures well known and described in the art.

### Methods of Preparation

The compounds of the invention may be prepared by conventional methods for chemical synthesis, e.g. those described in the working examples.

### Biological Activity

The compounds of the invention have been found to possess ion channel modulating activity as measured by standard electrophysiological methods. Due to their activity as modulators of ion channels, and in particular the potassium and chloride channels, the compounds of the invention are considered useful for the treatment of a wide range of diseases and conditions.

In a special embodiment, the compounds of the invention are considered useful for the treatment, prevention or alleviation of a bone metabolic disease, such as an osteoclast related bone disease, osteoporosis, postmenopausal osteoporosis, secondary osteoporosis, osteolytic breast cancer bone metastasis, osteolytic cancer invation, or Paget's disease of bone; or a disease that is responsive to inhibition of angiogenesis, such as diseases that involve the proliferation of tumor cells, cancer, metastatic cancer, prostate cancer, lung cancer, breast cancer, bladder cancer, renal cancer, colon cancer, gastric cancer, pancreatic cancer, ovarian cancer, melanoma, hepatoma, sarcoma, lymphoma; or an ophthalmic angiogenesis related diseases, such as exudative macular degeneration, age-related macular degeneration (AMD), retinopathy, diabetic retinopathy, proliferative diabetic retinopathy, diabetic macular edema (DME), ischemic retinopathy (e.g. retinal vain or artery occlusion), retinopathy of prematurity, neovascular glaucoma, and corneal neovascularization; or a disease, disorder or condition that is responsive to reduction of intraocular pressure, such as ocular hypertension, open-angle glaucoma, chronic open-angle glaucoma, angle-closure glaucoma and ciliary injection caused by angle-closure glaucoma; or rheumatoid arthritis, psoriasis and sickle-cell anaemia.

In another special embodiment, the compounds of the invention are considered useful for the treatment, prevention or alleviation of a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhoea, narcolepsy, sleeping disorders, sleep apnoea, Reynaud's disease, intermittent claudication, Sjögren's syndrome, xerostomia, arrhythmia, cardiovascular disorders, hypertension, myotonic dystrophy, myotonic muscle dystrophia, spasticity, xerostomia, diabetes Type II, hyperinsulinemia, premature labour, cancer, brain tumours, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma), baldness, cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy or cardiomyopathy.

In a more preferred embodiment, the compounds of the invention are considered useful for the treatment, prevention or alleviation of a respiratory disease, urinary incontinence, erectile dysfunction, anxiety, epilepsy, psychosis, schizophrenia, bipolar disorder, depression, amyotrophic lateral sclerosis (ALS), Parkinson's disease or pain.

In another more preferred embodiment, the compounds of the invention are considered useful for the treatment, prevention or alleviation of psychosis, schizophrenia, bipolar disorder, depression, epilepsy, Parkinson's disease or pain.

In a third more preferred embodiment, the compounds of the invention are considered useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In a fourth more preferred embodiment, the compounds of the invention are considered useful for the treatment, prevention or alleviation of cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy, cardiomyopathy or a genetic disease.

In a fifth more preferred embodiment, the compounds of the invention are considered useful for the treatment, prevention or alleviation of cardiac ischemia, ischemic heart disease, hypertrophic heart, cardiomyopathy or failing heart.

In a sixth more preferred embodiment, the compounds of the invention are considered useful for the treatment, prevention or alleviation of cardiac arrhythmia, atrial fibrillation and/or ventricular tachyarrhythmia.

In a seventh more preferred embodiment, the compounds of the invention are considered useful for the treatment, prevention or alleviation of schizophrenia, depression or Parkinson's disease.

In an eight more preferred embodiment, the compounds of the invention are considered useful for the treatment, prevention or alleviation of a sexual dysfunction, incl. male sexual dysfunction and female sexual dysfunction, and incl. male erectile dysfunction.

In an even more preferred embodiment the compound of the invention may be co-administered with a phosphodiesterase inhibitor, in particular a phosphodiesterase 5 (PDE5) inhibitor, e.g. sildenafil, tadalafil, vardenafil and dipyridamole, or with an agent that potentiates endothelium-derived hyperpolarizing factor-mediated responses, in particular calcium dobesilate or similar 2,5-dihydroxybenzenesulfonate analogs.

In a most preferred embodiment the compound of the invention is used in a combination therapy together with sildenafil, tadalafil, vardenafil or calcium dobesilate.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of a compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the compound of the invention together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé in powder, or in liquid form, and parenteral administration, in particular cutaneous, subcutaneous, intramuscular, or intravenous injection. The pharmaceutical composition of the invention can be manufactured by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Pharmaceutical Kits of Parts

According to the invention there is also provided a kit of parts comprising at least two separate unit dosage forms (A) and (B):

(A) a cinnamic amide derivative of the invention; and

(B1) a phosphodiesterase inhibitor; or

(B2) an agent that potentiates endothelium-derived hyperpolarizing factor-mediated responses; and optionally

(C) instructions for the simultaneous, sequential or separate administration of the cinnamic amide derivative of A, and the phosphodiesterase inhibitor of B1, or an agent that potentiates endothelium-derived hyperpolarizing factor-mediated responses of B2, to a patient in need thereof.

In a more preferred embodiment the phosphodiesterase inhibitor for use according to the invention (B1) is a phosphodiesterase 5 (PDE5) inhibitor, and in an even more preferred embodiment the phosphodiesterase inhibitor for use according to the invention is sildenafil, tadalafil or vardenafil.

In another more preferred embodiment the agent that potentiates endothelium-derived hyperpolarizing factor-mediated responses for use according to the invention (B2) is calcium dobesilate.

The cinnamic amide derivative of the invention and the phosphodiesterase inhibitor or the agent that potentiates endothelium-derived hyperpolarizing factor-mediated responses for use according to the invention may preferably be provided in a form that is suitable for administration in conjunction with the other. This is intended to include instances where one or the other of two formulations may be administered (optionally repeatedly) prior to, after, and/or at the same time as administration with the other component.

Also, the cinnamic amide derivative of the invention and the phosphodiesterase inhibitor or the agent that potentiates endothelium-derived hyperpolarizing factor-mediated responses for use according to the invention may be administered in a combined form, or separately or separately and sequentially, wherein the sequential administration is close in time or remote in time. This may in particular include that two formulations are administered (optionally repeatedly) sufficiently closely in time for there to be a beneficial effect for the patient, that is greater over the course of the treatment of the relevant condition than if either of the two formulations are administered (optionally repeatedly) alone, in the absence of the other formulation, over the same course of treatment. Determination of whether a combination provides a greater beneficial effect in respect of, and over the course of treatment of, a particular condition, will depend upon the condition to be treated or prevented, but may be achieved routinely by the person skilled in the art.

When used in this context, the terms “administered simultaneously” and “administered at the same time as” include that individual doses of the positive allosteric nicotine receptor modulator and the cognitive enhancer are administered within 48 hours, e.g. 24 hours, of each other.

Bringing the two components into association with each other, includes that components (A) and (B) may be provided as separate formulations (i.e. independently of one another), which are subsequently brought together for use in conjunction with each other in combination therapy; or packaged and presented together as separate components of a “combination pack” for use in conjunction with each other in combination therapy.

### Methods of Therapy

In another aspect the invention provides a method of treatment, prevention or alleviation of a disease, disorder or condition of a living animal body, including a human, which disorder, disease or condition is responsive to activation of an ion channel, and in particular a potassium channel or a chloride channel, which method comprises the step of administering to such a living animal body in need thereof, a therapeutically effective amount a compound capable of activating the ion channel, or a pharmaceutically-acceptable addition salt thereof.

The preferred medical indications contemplated according to the invention are those stated above.

It is at present contemplated that a suitable dosage of the active pharmaceutical ingredient (API) is within the range of from about 0.1 to about 1000 mg API per day, more preferred of from about 1 to about 500 mg API per day, most preferred of from about 1 to about 100 mg API per day, dependent, however, upon the exact mode of administration, the form in which it is administered, the indication considered, the subject and in particular the body weight of the subject involved, and further the preference and experience of the physician or veterinarian in charge.

### BRIEF DESCRIPTION OF THE DRAWING

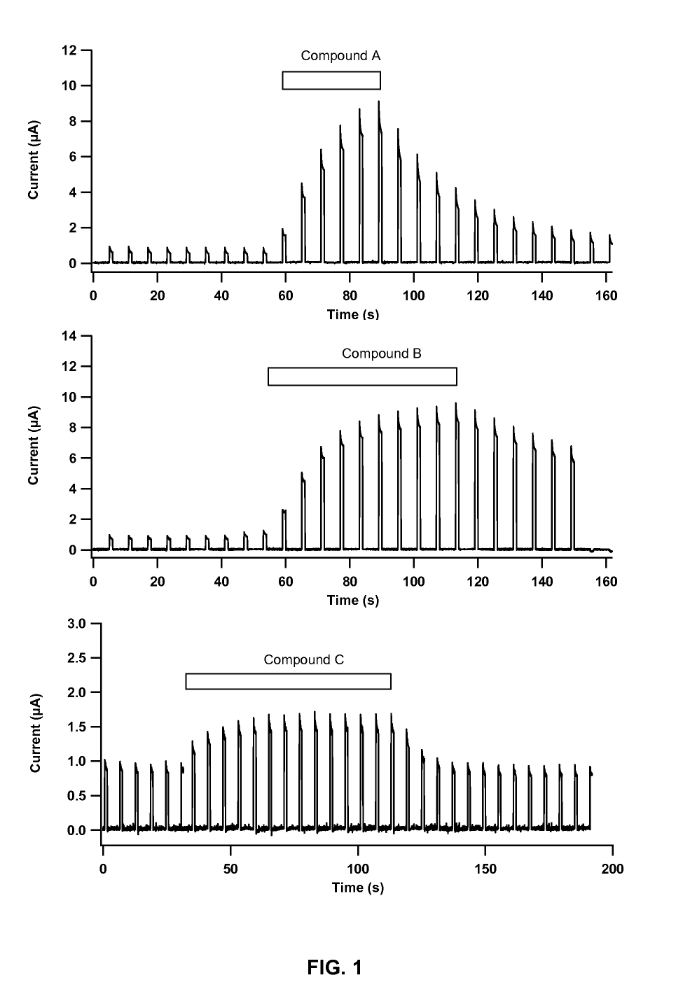

The present invention is further illustrated by reference to the accompanying drawing, in which FIG. 1 shows the BK channel opening activity [current (μA) vs. time (s)] of three cinnamic amide derivatives representative of the invention, i.e. Compound 22 (A), Compound 9 (B) and Compound 19 (C), determined by a standard electrophysiological method using BK channels heterologously expressed in Xenopus laevis oocytes.

### EXAMPLES

The invention is further illustrated with reference to the following examples, which are not intended to be in any way limiting to the scope of the invention as claimed.

### Example 1

### Preparatory Example

Abbreviations used herein:

AcOEt: ethyl acetate

CFM: chloroform

DCM: dichloromethane

DMA: dimethylacetamide

DMF: N,N-dimethylformamide

DMAP: 4-dimethylaminopyridine

DMSO: dimethylsulfoxide

EDC.HCl: 1-ethyl-3-(3-dimethylaminopropyl) carbodiimide hydrochloride

EtOH: ethanol

Hex: hexane

MEK: methylethylketone

MeOH: methanol

MgSO4: magnesium sulphate

PE: petroleum ether (fraction boiling at 40-60° C.)

Py: Pyridine

THF: tetrahydrofuran

TOL: toluene

### Synthesis of the Intermediate Compounds

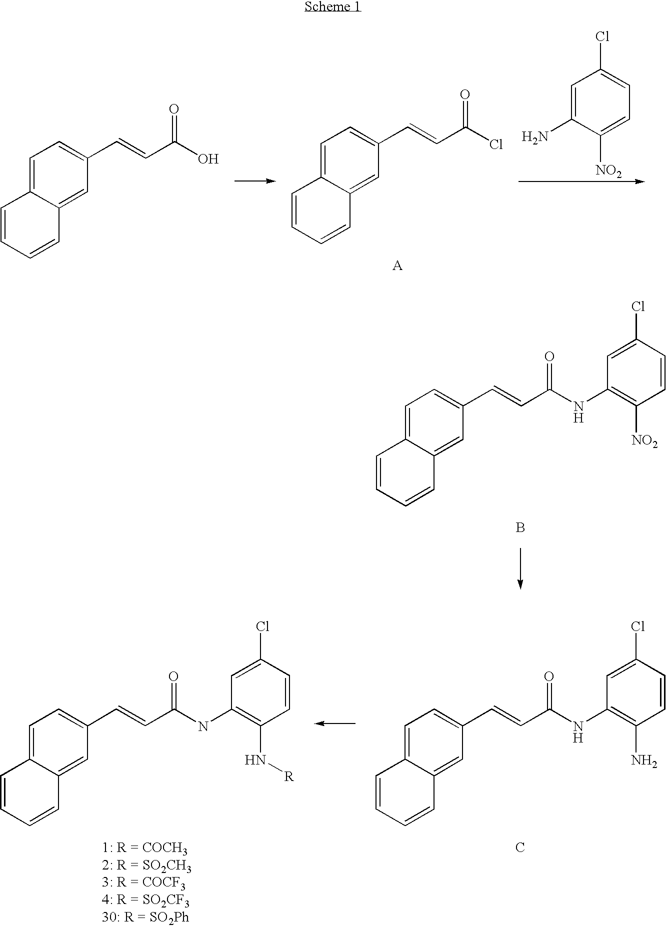

### (E)-3-Naphthalen-2-yl-acryloyl chloride (A, Scheme 1)

To a stirred suspension of 3-(2-naphthylacrylic acid) (2.27 g, 1.1 eq) in DCM, oxalyl chloride (1.3 ml, 1.3 eq) is added drop wise at 0° C., followed by 1-2 drops of dry DMF. The resulting yellow solution is allowed to reach room temperature spontaneously and then stirred at room temperature until starting material disappears completely on TLC (˜1 hour). The mixture is then evaporated to dryness under vacuum and the residue is taken up in DCM and washed with aqueous NaHCO₃. The organic phase, dried over magnesium sulphate and evaporated to dryness, gave 2.48 g (˜100% yield) of the title compound which is used as such for the next step without further purification.

### (E)-N-(5-Chloro-2-nitro-phenyl)-3-naphthalen-2-yl-acrylamide (B, Scheme 1)

To a solution of the Intermediate A (2.48 g, 1 eq) dissolved in DCM (20 ml), 1 eq of Py is added (˜0.9 ml) and 5-chloro-2-nitroaniline (2 g, 1 eq). More Py is immediately added (1.8 ml) and the resulting solution is stirred at room temperature overnight and under nitrogen. The reaction mixture is diluted with DCM (150 ml), washed with 1.5 N HCl (50 ml), water (50 ml) and brine (50 ml), dried and concentrated under vacuum to give the title compound as a yellow solid (3.4 g, 94% yield). This crude material is purified by crystallization from AcOEt.

### (E)-N-(2-Amino-5-chloro-phenyl)-3-naphthalen-2-yl-acrylamide (C, Scheme 1)

To a stirred suspension of the Intermediate C (2 g, 1 eq) in MeOH (75 ml), zinc dust is added (3.60 g, 10 eq) and the suspension is cooled to 0° C. To this suspension, ammonium formate is added portion wise (3.467 g, 10 eq). The reaction mixture is allowed to reach room temperature spontaneously, stirred at room temperature for 2 further hours and finally diluted with THF (100 ml). The mixture is filtered through celite, the filtrate evaporated to dryness, and the resulting brown oily liquid is purified by flash chromatography using silica gel (230-400 mesh) and 5%-20% AcOEt in Hex as eluent, to afford the title compound as a brownish solid (1.3 g, 73%).

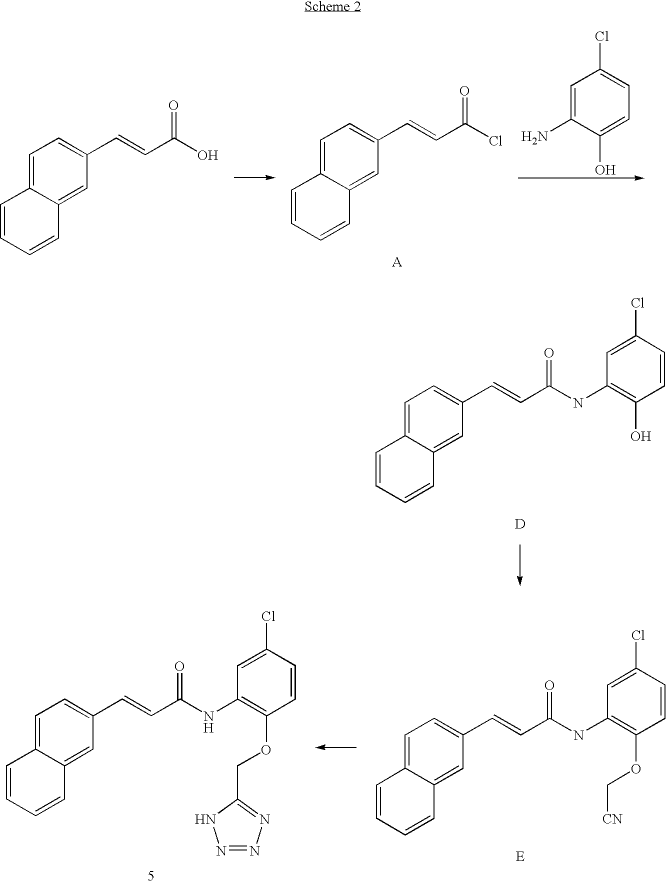

### (E)-N-(5-Chloro-2-hydroxy-phenyl)-3-naphthalen-2-yl-acrylamide (D, Scheme 2)

To an ice-cooled and stirred solution of the Intermediate A (1.09 g, 1 eq) in THF (10 ml), a solution of 2-amino-4-chlorophenol (0.72 g, 1 eq) in 1N NaOH (10 ml) is added. By addition of 1N NaOH, the ice-cooled mixture is kept at pH in between 7.5 and 8.5 for 30 min, and then stirred for an additional hour at room temperature. The solvent is evaporated under reduced pressure, and water (15 ml) is added to the residue. The resulting solution is acidified with 1N HCl (pH ˜5) and the solid precipitated is filtered, washed with water and dried to give a brown solid (1.3 g, yield 81%). This crude material is purified by flash chromatography using silica gel (60-120 mesh) and AcOEt (0-40%) in Hex.

### (E)-N-(5-Chloro-2-cyanomethoxy-phenyl)-3-naphthalen-2-yl-acrylamide (E, Scheme 2)

A mixture of Intermediate D (0.4 g, 1 eq), sodium iodide (0.22 g, 1.2 eq), potassium carbonate (0.205, 1.2 eq), and chloroacetonitrile (0.112 g, 1.2 eq) in MEK is refluxed for 2 days. The reaction mixture is diluted with AcOEt (40 ml), washed with 1.5 N HCl (20 ml), water (20 ml) and brine (20 ml), dried and evaporated to dryness to give a brown solid (0.31 mg, 69% yield). This crude material is purified by flash chromatography using 230-400 mesh silica gel and by elution with AcOEt (22%) in Hex (0.07 g).

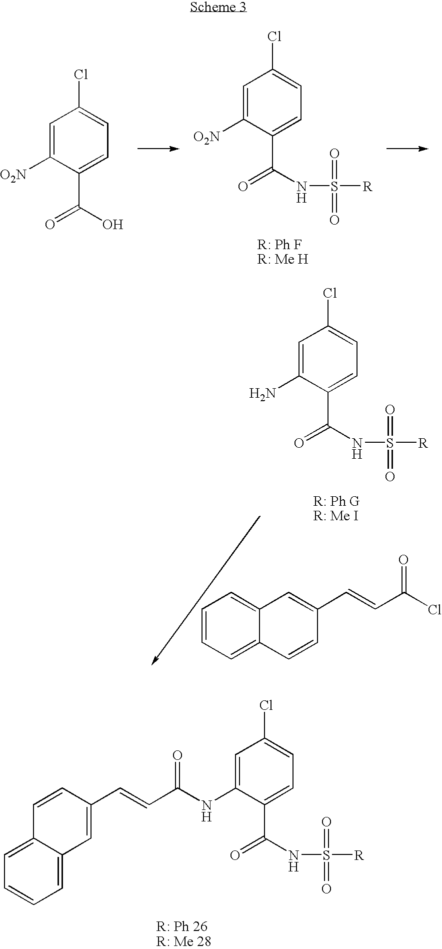

### N-(2 Amino-4-chloro-benzoyl)-benzenesulfonamide (F, Scheme 3)

Commercial 4-chloro-2-nitrobenzoic acid (2 g, 1 eq) is added to a stirred solution of benzenesulfonamide (1.56 g, 1 eq), DMAP (3.64 g, 3 eq) and EDC.HCl (3.80 g, 2 eq) in dry DCM (70 ml). After overnight stirring at room temperature, the reaction mixture is diluted with DCM (150 ml), washed with 1.5 HCl (50 ml), water (50 ml) and brine (50 ml), dried and evaporated to dryness, in order to afford the title compound as an off-white solid (3.1 g, 93% yield; 93% purity at HPLC).

### N-(2-Amino-4-chloro-benzoyl-benzenesulfonamide (G, Scheme 3)

Raney-nickel (0.15 g) is carefully added to a solution of the Intermediate F (1 g, 1 eq) in MeOH (40 ml). The reaction mixture is hydrogenated overnight, filtered through celite and the filtrate is evaporated to dryness to give the title compound as an off-white solid (0.8 g, 94%).

### N-(4-Chloro-2-nitro-benzoyl)-methanesulfonamide (H, Scheme 3)

To a suspension of 4-chloro-2-nitrobenzoic acid (5 g, 1 eq) in DCM (100 ml), EDC.HCl (9.5 g, 2 eq) and DMAP (9 g, 3 eq) are added. The resulting brown solution is stirred for 10 min and methanesulfonamide (2.35 g, 1 eq) is then added. The reddish solution is stirred at room temperature overnight, diluted with DCM (100 ml), washed with 1.5 N HCl (2×50 ml) and water (50 ml), dried and evaporated to dryness, to give a yellowish solid (4.6 g, 67% yield). This crude material is suspended in DCM (15 ml), stirred for 15 min, filtered and dried, to afford the title compound as white solid (2 g, 29%, purity 98%), which is used as such for the next step.

### N-(2-Amino-4-chloro-benzoyl)-methanesulfonamide (I, Scheme 3)

To a solution of the Intermediate H (1 g, 1 eq) in MeOH (80 ml), Raney-nickel (0.2 g) is carefully added. The reaction mixture is hydrogenated for 7 hours and then filtered through celite. The celite is washed with MeOH (50 ml) and the filtrate is evaporated to give the title compound as an off-white solid (0.8 g, 90% yield; 84% purity). The compound, as such, is used for the next step without further purification.

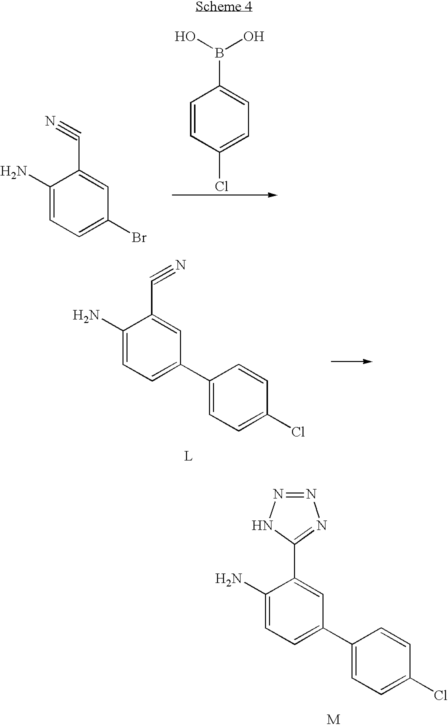

### 4-Amino-4′-chloro-biphenyl-3-carbonitrile (L, Scheme 4)

To a mixture of 2-Amino-5-bromo-benzonitrile⁴ (5.5 g, 1 eq), 4-chlorobenzeneboronic acid (4.8 g, 1.1 eq), potassium carbonate (12.7 g, 3.3 eq), dimethoxy ethane (80 ml) and water (40 ml), bistriphenylphosphine palladium (II) chloride (0.2 g) is added. The resulting mixture is refluxed for 24 hours and then evaporated to dryness. The residue is purified by flash chromatography using DCM as eluent (5.32 g, 83% yield).

### 4′-Chloro-3-(1H-tetrazol-5-yl)-biphenyl-4-ylamine (M, Scheme 4)

A mixture of 4-Amino-4′-chloro-biphenyl-3-carbonitrile (5.3 g, 1 eq), sodium azide (2.3 g, 1.5 eq), trethylamine hydrochloride (4.9 g, 1.5 eq) is suspended in 40 ml of toluene and heated (60° C.) overnight. To the reaction mixture, cooled to room temperature, water and 4M HCl are added, to afford the title compound as a white precipitate. This was collected by filtration (4.83 g, 77% yield) and used for the next step without further purification.

### Synthesis of Compounds of the Invention

### (E)-N-(2-Acetylamino-5-chloro-phenyl)-3-naphthalen-2-yl-acrylamide (1, Scheme 1)

To a stirred and ice-cooled suspension of the Intermediate B (0.35 g, 1 eq) in Py (5 ml), acetic anhydride (0.10 ml, 1 eq) is added drop wise and the resulting mixture is first allowed to reach room temperature spontaneously and then stirred at room temperature overnight. Evaporation to dryness of the resulting mixture provides a solid residue which is first triturated with water, filtered and finally dried, to give the title compound as a yellow solid (0.34 g, 86% yield). This crude product is purified by crystallisation from CFM/MeOH, to give a yellow crystalline solid (0.15 mg). M.p. 240.1-247.3° C.

### (E)-N-(5-Chloro-2-methanesulfonylamino-phenyl-3-naphthalen-2-yl-acrylamide (2, Scheme 1)

To a stirred and ice-cooled suspension of Intermediate B (0.35 g, 1 eq) in Py (5 ml), methanesulphonyl chloride (0.086 ml, 1 eq) is added drop wise. The reaction mixture is allowed to reach room temperature spontaneously, then stirred at room temperature overnight and finally evaporated to dryness under reduced pressure. The residue is triturated with water (10 ml), stirred for 20 min, filtered and dried to give a brown solid (0.355 g, yield 82%). The new residue is purified by crystallisation from a mixture of CFM/MeOH. M.p. 179.4-184.1° C.

### (E)-N-[5-Chloro-2-(2,2,2-trifluoro-acetylamino)-phenyl]-3-naphthalen-2-yl-acrylamide (3, Scheme 1)

To a stirred and ice-cooled suspension of Intermediate B (0.5 g, 1 eq) in Py (10 ml), trifluoroacetic anhydride (0.22 ml, 1 eq) is added drop wise and the resulting mixture is first allowed to reach room temperature spontaneously and then stirred at room temperature overnight. Evaporation to dryness of the resulting mixture provides a solid residue which is first triturated with water (10 ml), stirred for 20 min, filtered and finally dried, to give the crude compound as a brown solid (0.6 g, ˜92% yield). This latter is purified by flash chromatography, using 8% AcOEt in Hex as eluent to give the title compound as a yellow solid (0.06 g, Rf: 0.31). M.p. 179.3-185.3° C.

### (E)-N-(5-Chloro-2-trifluoromethanesulfonylamino-phenyl-3-naphthalen-2-yl-acrylamide (4, Scheme 1)

To a stirred and ice-cooled suspension of Intermediate B (0.25 g, 1 eq) in DMAP (0.28 g, 3 eq) and DCM (15 ml), trifluoromethanesulphonyl chloride is added drop wise. The mixture is allowed to reach room temperature spontaneously and then it is stirred at room temperature for further 5 hours. The reaction mixture is then diluted with DCM (50 ml), washed with water (25 ml) and brine (25 ml), dried over Magnesium sulphate, filtered and evaporated to dryness to give a brown solid (0.3 g, 85% yield). This latter is purified by flash chromatography using ethyl acetate (10-40%) in Hex as eluent, to give the title compound as a brownish solid (0.05 g). LC-ESI-HRMS of [M+H]+ shows 455.045 Da. Calc. 455.044401 Da, dev. 1.3 ppm.

### (E)-N-[5-Chloro-2-(1H-tetrazol-5-ylmethoxy)-phenyl]-3-naphthalen-2-yl-acrylamide (5, Scheme 2)

A suspension of Intermediate E (0.125 g, 1 eq), NaN₃ (0.0448 g, 2 eq), ammonium chloride (0.0376 g, 2 eq) in DMF (2 ml) is heated at 120° C. for 6 hours. The reaction mixture is cooled to room temperature and the solvent is evaporated under vacuum, to give a brown gummy residue. This residue is suspended in a mixture of water (4 ml) and 1.5 N HCl (1 ml), and the suspension stirred at room temperature for 30 min. The solid is then collected by filtration, washed with water and diethyl ether and dried, to give the title compound as a brown solid (0.11 g, 72% yield). M.p. 218-223° C.

### (E)-N-(5-Chloro-2-hydroxy-phenyl)-3-(3-nitro phenyl)-acrylamide (6)

To a solution of commercial 3-nitrocinnamic acid (1.19 g, 1 eq) in dry TOL (40 ml), oxalyl chloride (5.37 ml, 10 eq) is carefully added, followed by 1-2 drops of dry DMF. The resulting yellow solution is refluxed for 3 hours and then evaporated to dryness, to get the 3-nitro cinnamoyl chloride as a solid residue (1.3 g, yield ˜100%). To a solution of commercial 5-chloro-2-hydroxyaniline (0.88 g, 1 eq) in dry TOL (30 ml), a solution of the above acid chloride in dry TOL (10 ml) is added drop wise and the mixture is heated with stirring for 24 hours. The day after the suspension is evaporated to dryness (1.9 g, yield 97%) and the resulting solid residue is purified by crystallisation from EtOH. LC-ESI-HRMS of [M+H]+ shows 319.0487 Da. Calc. 319.048561 Da, dev. 0.4 ppm.

### (E)-3-(3,5-Bis-trifluoromethyl-phenyl-N-(5-chloro-2-hydroxy-phenyl-acrylamide (7)

To a solution of commercial 3,5-bis(trifluoromethyl)cinnamic acid (0.45 g, 1 eq) in dry TOL (20 ml), oxalyl chloride (1.4 ml, 10 eq) is carefully added, followed by 1-2 drops of dry DMF. The resulting yellow solution is refluxed for 3 hours and then evaporated to dryness, to get the 3,5-bis(trifluoromethyl)cinnamoyl chloride as a yellow oil (0.48 g, yield ˜100%). To a solution of commercial 5-chloro-2-hydroxyaniline (0.227 g, 1 eq) in dry TOL (20 ml), a solution of the above acid chloride in dry TOL (5 ml) is added drop wise and the mixture is stirred at room temperature overnight. The day after the suspension is evaporated to dryness (0.62 g, yield ˜97%) and the resulting solid residue is purified by crystallisation from EtOH. LC-ESI-HRMS of [M−H]− shows 408.0231 Da. Calc. 408.0226 Da, dev. 1.2 ppm.

### (E)-N-[5-Chloro-2-(1H-tetrazol-5-yl-phenyl]-3-(4-fluoro-3-trifluoromethyl-phenyl) -acrylamide (8)

To a solution of commercial 4-fluoro-3-(trifluoromethyl)cinnamic acid (0.45 g, 1 eq) in dry TOL (20 ml), oxalyl chloride (1.7 ml, 10 eq) is carefully added, followed by 1-2 drops of dry DMF. The resulting yellow solution is refluxed for 3 hours and then evaporated to dryness, to get the 4-fluoro-3-(trifluoromethyl)cinnamoyl chloride as a solid residue (0.48 g, yield ˜100%). To a solution of 5-(2-amino-4-chlorophenyl)tetrazole prepared as described by Valgeirsson et al. in Journal of Medicinal Chemistry 2004 47 (27) 6948-6957 (0.34 g, 1 eq) in dry TOL (30 ml) and Py (1.5 ml), a solution of the above acid chloride in dry TOL (5 ml) is added drop wise and the mixture is stirred at room temperature overnight. The day after the suspension is evaporated to dryness (0.7 g, yield 89%) and the resulting solid residue is purified by crystallisation from a mixture of DMSO and water. LC-ESI-HRMS of [M+H]+ shows 412.0578 Da. Calc. 412.058825 Da, dev. −2.5 ppm.

### (E)-N-[5-Chloro-2-(1H-tetrazol-5-yl)-3-phenyl]-3-naphthalen-2-yl-acrylamide (9)

A solution of Intermediate A (0.22 g, yield ˜100%) in dry TOL (10 ml) is added drop wise to a mixture of 5-chloro-2-(1H-tetrazol-5-yl)-phenylamine prepared as described by Valgeirsson et al. in Journal of Medicinal Chemistry 2004 47 (27) 6948-6957 (0.199 g, 1 eq) in Py (1 ml) and dry TOL (5 ml), and stirring is continued overnight at room temperature. The solvent is then evaporated in vacuo and the residue is suspended in diluted HCl. The residue is collected with by filtration, washed with water, dried (0.30 g, ˜80%) and purified by crystallisation from EtOH/DMSO. LC-ESI- HRMS of [M−H]− shows 374.0805 Da. Calc. 374.080863 Da, dev. −1 ppm.

### (E)-N-[5-Chloro-2-(1H-tetrazol-5-yl)-phenyl]-3-(3,4-dichloro-phenyl)-acrylamide (10)

Commercial 3,4-dichlorocinnamic acid (1 g, 1 eq) is dissolved in an excess of thionyl chloride (3.3 ml) and dry TOL (25 ml), followed by 1-2 drops of dry DMF. The resulting yellow solution is refluxed until the starting material disappears completely and then it is evaporated to dryness. The residue is taken up in DCM, washed with aqueous NaHCO₃, dried over magnesium sulphate and evaporated to dryness, to get the (E)-3-(3,4-dichloro-phenyl)-acryloyl chloride as a yellow oil (1.08 g, yield ˜100%). This latter is dissolved again dry TOL (10 ml) and its solution is added drop wise to a mixture of 5-chloro-2-(1H-tetrazol-5-yl)-phenylamine prepared as described by Valgeirsson et al. in Journal of Medicinal Chemistry 2004 47 (27) 6948-6957 (0.440 g. 1 eq) in dry TOL (15 ml) and Py (1 ml), and stirring is continued overnight. The solvent is finally evaporated in vacuo and the residue is suspended in diluted HCl. The residue is collected by filtration, washed with water, dried (1.80 g, ˜100%) and purified by crystallisation from a mixture of DMSO and water. LC-ESI-HRMS of [M+H]+ shows 394.0024 Da. Calc. 394.002919 Da, dev. −1.3 ppm.

### (E)-N-[4-Bromo-2-(1H-tetrazol-5-yl)-phenyl]-3-(3-trifluoromethyl-phenyl)-acrylamide (11)

To an iced-cooled solution of 5-(2-amino-5-bromophenyl)tetrazole prepared as described by in US 2002037905 (0.5 g, 1 eq) in Py (6 ml), commercial trans-3-(trifluoromethyl)cinnamoyl chloride (0.5 g, 1 eq) is added and stirring continued at room temperature for 24 hours. The resulting solution is evaporated to dryness and resulting crude pale yellow solid (yield ˜100%) is purified by crystallisation from a mixture of AcOEt and PE to get 0.13 g (yield 15%) of the title compound. LC-ESI-HRMS of [M−H]− shows 436.0018 Da. Calc. 436.002082 Da, dev. −0.6 ppm.

### (E)-3-(3,5-Bis-trifluoromethyl-phenyl)-N-[4′-chloro-3-(1H-tetrazol-5-yl)-biphenyl-4-yl]-acrylamide (12)

A solution of (E)-3-(3,5-bis-trifluoromethyl-phenyl)-acryloyl chloride (prepared as described for 7) (0.426 g, 1 eq) in TOL (10 ml) is added to a solution of an equimolar amount of Intermediate M (0.38 g, 1 eq) in a dry TOL (15 ml) and Py (1 ml) and the reaction mixture is refluxed for 4 hours. After cooling, the solvent is evaporated in vacuo and the residue is suspended in diluted HCl. The solid residue is collected by filtration, washed with water and finally purified by crystallization from EtOH (0.63 g, 83%). LC-ESI-HRMS of [M+H]+ shows 538.0882 Da. Calc. 538.086931 Da, dev. 2.4 ppm.

### (E)-3-(3,5-Bis-trifluoromethyl-phenyl)-N-[4-bromo-2-(1H-tetrazol-5-yl)-phenyl]-acrylamide (13)

A solution of (E)-3-(3,5-bis-trifluoromethyl-phenyl)-acryloyl chloride (prepared as described for 7) (0.5 g, 1 eq) in TOL (15 ml) is added to a mixture of 4-bromo-2-(1H-tetrazol-5-yl)-phenylamine prepared as described by in US 2002037905 (0.39 g, 1 eq) in a mixture of dry TOL (15 ml) and Py (2 ml) and stirring is continued overnight. The solvent is evaporated in vacuo and the residue is suspended in diluted aqueous HCl. The solid residue is collected by filtration, washed with water, dried (0.76 g, 80%) and purified by crystallisation from DMA/water. LC-ESI-HRMS of [M−H]− shows 503.9894 Da. Calc. 503.989466 Da, dev. −0.1 ppm.

### (E)-N-[4-Bromo-2-(1H-tetrazol-5-yl)-phenyl]-3-naphthalen-2-yl-acrylamide (14)

A solution of the Intermediate A (0.49 g, 1 eq) in dry TOL (15 ml) is added to a solution of an equimolar amount of 4-bromo-2-(1H-tetrazol-5-yl)-phenylamine prepared as described by in US 2002037905 (0.545 g) in a mixture of dry TOL (15 ml) and Py (2 ml) and stirring is continued overnight. The solvent is evaporated in vacuo and the residue is suspended in diluted aqueous HCl. The residue is collected by filtration, washed with water, dried (0.76 g, 80%) and purified by crystallisation from DMA/water. LC-ESI-HRMS of [M−H]− shows 418.0294 Da. Calc. 418.030348 Da, dev. −2.3 ppm.

### 4-Chloro-2-(E)-3-naphthalen-2-yl-acryloylamino)-benzoic acid (15)

A solution of the Intermediate A (0.38 g, 1 eq) in dry TOL (10 ml) is added drop wise to a mixture of commercial 2-amino-4-chlorobenzoic acid (0.30 g, 1 eq) in dry TOL (10 ml) and Py (1.5 ml), and stirring is continued overnight. To this suspension, HCl 1N is added (pH ˜5) and stirring is continued for 10 min. The two phases are separated and the organic phase is washed repeatedly with water, dried over magnesium sulphate and evaporated to dryness. The resulting solid residue is washed with PE and dried under vacuum overnight (0.16 g, 26%). LC-ESI-HRMS of [M+H]+ shows 352.0723 Da. Calc. 352.074047 Da, dev. −5 ppm.

### 4-Chloro-2-[(E)-3-(3,4-dichloro-phenyl-acryloylamino]-benzoic acid (16)

Commercial (E)-3-(3,4-dichloro-phenyl)-acrylic acid (0.56 g, 1 eq) is dissolved in an excess of oxalyl chloride (2 ml) and dry DCM (20 ml), followed by 1-2 drops of dry DMF. The resulting yellow solution is refluxed until the starting material disappears completely and then it is evaporated to dryness. The residue is taken up in DCM, washed with aqueous NaHCO₃, dried over magnesium sulphate and evaporated to dryness, to get the (E)-3-(3,4-cichloro-phenyl)-acryloyl chloride (yield ˜100%). This latter is dissolved again dry DCM (5 ml) and its solution is added drop wise to a solution of ice-cooled commercial 2-amino-4-chlorobenzoic acid (0.44 g, 1 eq) in dry DCM (3 ml) and Py (2 ml), and stirring is continued overnight. To the resulting suspension, an equivolume amount of diluted HCl 1N is added (pH ˜5) and stirring is continued for 10 min. The two phases are separated and the organic phase is washed repeatedly with water, dried over magnesium sulphate and evaporated to dryness. The resulting solid residue is washed with PE, dried under vacuum overnight (0.72 g, 75%) and recrystallised from a mixture AcOEt/PE. LC-ESI-HRMS of [M−H]− shows 367.9636 Da. Calc. 367.964803 Da, dev. −3.3 ppm.

### (E)-3-Naphthalen-2-yl-N-[2-(2,2,2-trifluoro-1-hydroxy-1-trifluoromethyl-ethyl)-phenyl]-acrylamide (17)

A solution of Intermediate A (0.2 g, 1 eq) in dry DCM (2 ml) is added drop wise to an ice-cooled solution of commercial 2-(hexafluoro-2-hydroxyisopropyl)aniline (0.74 g, 1 eq) in dry DCM (3 ml) and Py (2 ml) and the mixture is stirred overnight at room temperature. Water and HCl 1N are then added until pH 4-5, and the organic phase is separated and washed again with water, dried and evaporated to dryness. The resulting solid is washed with PE, dried (˜0.7 g, yield 70%) and recrystallised from a solution of DCM/PE. LC-ESI-HRMS of [M+H]+ shows 440.1096 Da. Calc. 440.108522 Da, dev. 2.4 ppm.

### (E)-3-(3,5-Bis-trifluoromethyl-phenyl)-N-[4-iodo-2-(1H-tetrazol-5-yl)-phenyl]-acrylamide (18)

To a solution of 0.5 g (1 eq) of (E)-3-(3,5-bis-trifluoromethyl-phenyl)-acryloyl chloride (prepared as described in 13) in dry TOL (15 ml), a solution of 4-iodo-2-(1H-tetrazol-5-yl)-phenylamine prepared as described in WO 2004018461 (0.47 g, 1 eq) in TOL (15 ml) and Py (1 ml) is added drop wise. Stirring is continued at room temperature for 5 hours and the resulting suspension is evaporated to dryness. The solid residue is washed with water, dried (0.749 g, 82% yield) and purified by prep LCMS. LC-ESI-HRMS of [M−H]− shows 551.9746 Da. Calc. 551.975628 Da, dev. −1.9 ppm.

### 4-Chloro-2-((E)-3-naphthalen-2-yl-acryloylamino)-benzenesulfonic acid (19)

To an ice-cooled solution of Intermediate A (0.27 g, 1 eq) in THF (5 ml), solution of 2-amino-4-chloro-benzenesulfonic acid prepared as described by Valgeirsson et al. in Journal of Medicinal Chemistry 2004 47 (27) 6948-6957 (0.235 g, 0.9 eq) in 1N NaOH (5 ml) is added. The solution is kept stirring at 0° C. for 15 min and at pH 7.5-8.5 and overnight at room temperature. THF is evaporated, and the resulting aqueous solution is acidified to pH ˜5 to give a white precipitate. This is collected by filtration and dried (0.26 g, yield 56%). The middle spot on the TLC (Rf=0.13) is isolated by flash chromatography by 8% MeOH in CFM as eluent, to give the title compound as a white solid (0.05 g). LC-ESI-HRMS of [M+H]+ shows 388.0421 Da. Calc. 388.041033 Da, dev. 2.7 ppm.

### 2-((E)-3-Biphenyl-4-yl-acryloylamino)-4-chloro-benzenesulfonic acid (20)

To a stirred and ice-cooled suspension of commercial 4-phenylcinnamic acid (0.25 g, 1 eq) in DCM, an excess of oxalyl chloride (0.5 ml) is added drop wise, followed by two drops of DMF. The reaction mixture is allowed to reach room temperature spontaneously, and then stirred at room temperature for an additional hour, when the starting material is completely disappeared on TLC. To the above reaction mixture, Py (0.1 ml) is added, followed by 2-amino-4-chloro-benzenesulfonic acid (prepared as described in 19). Immediately other 0.2 ml of Py are added and the resulting mixture is stirred at room temperature overnight and then evaporated to dryness. This residue is triturated with water (20 ml), stirred for 20 min and filtered. The filtered cake is washed with water several times with water and dried to give an off-white solid (0.35 g, 76%). The crude material is purified by flash chromatography using 0-6% MeOH in CFM as eluent, to afford the title compound as off white solid (0.13 mg). LC-ESI-HRMS of [M+H]+ shows 414.0573 Da. Calc. 414.056683 Da, dev. 1.5 ppm.

### (E)-N-(5-Chloro-4-fluoro-2-sulfamoyl-phenyl)-3-naphthalen-2-yl-acrylamide (21)

A solution of Intermediate A (0.55 g, 1 eq) in THF (10 ml), a mixture of 2-amino-4-chloro-5-fluoro-benzenesulfonamide prepared as described by Bierbaum et al. in Journal of Medical Chemistry 1963 6 (3) 272-275 (0.566 g, 1 eq) and 1N NaOH (5 ml) is added. The stirred mixture is kept at pH 7.5-8.5 with 1N NaOH for 30 min under ice-cooling and for an additional hour at room temperature. THF is evaporated under reduced pressure, and the residue is diluted with water (5 ml) and acidified with HCl 1N (pH ˜5). The solid precipitated is filtered, washed with water and dried to give a purple solid (0.41, yield ˜40%). The crude material is purified by flash chromatography using 20% AcOEt in Hex as eluent, to afford the title compound as an off white solid (0.075 g). LC-ESI-HRMS of [M−H]− shows 403.031 Da. Calc. 403.031945 Da, dev. −2.3 ppm.

### 6-Chloro-2H-chromene-3-carboxylic acid [5-chloro-2-(1H-tetrazol-5-yl)-phenyl]-amide (22)

To a solution of commercial 5-chloro(2H)-1-benzopyran-3-carboxylic acid (0.40 g, 1 eq) in TOL dry (20 ml), thionyl chloride (2.77 ml, 20 eq) is added, followed by 1-2 drops of dry DMF. The resulting yellow solution is refluxed for 3 hours and evaporated to dryness, to get the correspondent 6-chloro-2H-chromene-3-carbonyl chloride (yield ˜100%). To a solution of 5-chloro-2-(1H-tetrazol-5-yl)-phenylamine prepared as described by Valgeirsson et al. in Journal of Medicinal Chemistry 2004 47 (27) 6948-6957 in dry TOL (15 ml) and Py (1.5 ml, ˜10 eq), a solution of the above acid chloride derivative (1 eq) in dry TOL is added drop wise, and the resulting mixture is stirred at room temperature overnight. The day after the suspension is evaporated to dryness (˜0.73 g, ˜100% yield). The crude compound is easily purified by crystallization from EtOH. LC-ESI-HRMS of [M−H]− shows 386.0197 Da. Calc. 386.021156 Da, dev. −3.8 ppm.

### 5-Chloro-1H-indole-2-carboxylic acid [4′-fluoro-3-(1H-tetrazol-5-yl)-biphenyl-4-yl]amide (23)

To a solution of commercial 5-chloroindole-2-carboxylic-acid (0.5 g, 1 eq) in dry TOL (10 ml), thionyl chloride (1.86 ml, 10 eq) is carefully added, followed by 1-2 drops of dry DMF. The resulting yellow solution is refluxed for 4 hours and then evaporated to dryness, to get the 5-chloro-1H-indole-2-carbonyl chloride as a yellow residue (yield 100%). To a solution of 4′-fluoro-3-(1H-tetrazol-5-yl)-biphenyl-4-ylamine prepared as described in WO 2006064015 (0.65 g, 1 eq) in dry TOL (20 ml) and Py (3 ml), a solution of the above acid chloride in dry TOL (5 ml) is added drop wise and the mixture is heated (60° C.) overnight. The day after the suspension is evaporated to dryness (0.79 g, yield ˜72%) and the resulting solid residue is purified by crystallization from EtOH. LC-ESI-HRMS of [M−H]− shows 431.084 Da. Calc. 431.08234 Da, dev. 3.9 ppm.

### 6-Chloro-2H-chromene-3-carboxylic acid [4-bromo-2-(1H-tetrazol-5-yl)-phenyl]-amide (24)

A mixture of commercial 5-chloro(2H)-1-benzopyran-3-carboxylic acid (0.5 g, 1 eq) and an excess of thionyl chloride (5.6 ml) in dry TOL (20 ml) is heated (60-70° C.) until the starting material disappears completely. The mixture is then evaporated to dryness and the residue is taken up in DCM and washed with aqueous NaHCO₃. To the residue of the organic phase, dried over magnesium sulphate and evaporated to dryness, TOL and an equimolar amount of 4-bromo-2-(1H-tetrazol-5-yl)-phenylamine prepared as described by in US 2002037905 (0.95 g) in a mixture of anhydrous TOL (20 ml) and Py (2 ml) is added and stirring is continued overnight. The solvent is evaporated in vacuo and the residue is suspended in diluted HCl. The residue is collected with by filtration, washed with water, dried (0.80 g, ˜80% yield) and purified by crystallisation from EtOH. LC-ESI-HRMS of [M+H]+ shows 431.9869 Da. Calc. 431.986291 Da, dev. 1.4 ppm.

### 5-Chloro-1H-indole-2-carboxylic acid [5-chloro-2-(1H-tetrazol-5-yl)-phenyl]-amide (25)

To a solution of commercial 5-chloroindole-2-carboxylic-acid (0.50 g, 1 eq) in dry TOL (20 ml), an excess of oxalyl chloride (0.9 ml) is added, followed by 1-2 drops of dry DMF. The resulting yellow solution is heated (60° C.) for 3 hours and evaporated to dryness, to get the correspondent 5-chloro-1H-indole-2-carbonyl chloride (yield 100%). To a solution of 5-chloro-2-(1H-tetrazol-5-yl)-phenylamine prepared as described by Valgeirsson et al. in Journal of Medicinal Chemistry 2004 47 (27) 6948-6957 in dry TOL (20 ml) and Py (2 ml), a solution of the above acid chloride derivative in dry TOL (10 ml) is added drop wise and stirred at room temperature overnight. The day after the suspension is evaporated to dryness (˜0.73 g, ˜100% yield) and the yellow residue is suspended in HCl 1N, stirred for 20 min and filtered. The residue on the filter is repeatedly washed with water and finally dried (0.74 g, 78%). The crude compound is easily purified by crystallization from a mixture of DMSO and water. LC-ESI-HRMS of [M+H]+ shows 373.0389 Da. Calc. 373.03714 Da, dev. 4.7 ppm.

### (E)-N-(2-Benzenesulfonylaminocarbonyl-5-chloro-phenyl)-3-naphthalen-2-yl-acrylamide (26)

To a solution of 3-(2-naphthyl)acrylic acid (0.5 g, 1 eq), EDC.HCl (0.967 g, 2 eq) and DMAP (0.924 g, 3 eq) in DCM (20 ml), Intermediate G (0.78 g, 1 eq) is added and the resulting mixture is stirred overnight at room temperature. This is then diluted with DCM (30 ml), washed with 1.5 N HCl (2×20 ml), water (15 ml) and brine (15 ml), dried and evaporated to dryness to give a brown solid (1.1 g, 89% yield). The crude material is purified by flash chromatography using silica gel (230-400 mesh) and 10-40% AcOEt in Hex as eluent, to afford a white solid (0.4 g) which is further purified by crystallisation from MeOH/CFM. M.p. 328-332° C.

### 6-Bromo-2-oxo-2H-chromene-3-carboxylic acid [5-chloro-2-(1H-tetrazol-5-yl)-phenyl]-amide (27)

To a solution of 6-bromo-2-oxo-2H-chromene-3-carboxylic acid (1.31 g, 1 eq) in TOL dry (20 ml), an excess of oxalyl chloride (1.5 ml) is added, followed by 1-2 drops of dry DMF. The resulting solution is heated (60° C.) for 3 hours and evaporated to dryness, to get the correspondent 6-bromo-2-oxo-2H-chromene-3-carbonyl chloride (1.4 g, yield 100%). This latter is dissolved again in dry TOL (10 ml) and its solution is added drop wise to a mixture of 5-chloro-2-(1H-tetrazol-5-yl)-phenylamine prepared as described by Valgeirsson et al. in Journal of Medicinal Chemistry 2004 47 (27) 6948-6957 (0.95 g, 1 eq) in dry TOL (25 ml) and Py (4 ml), and stirring is continued overnight at 80° C. The solvent is finally evaporated in vacuo and the residue is suspended in water, collected by filtration, washed with water, dried and purified by crystallisation from MeOH (0.46 g, ˜30% yield). LC-ESI-HRMS of [M+H]+ shows 445.9646 Da. Calc. 445.965556 Da, dev. −2.1 ppm.

### (E)-N-(5-Chloro-2-methanesulfonylaminocarbonyl-phenyl-3-naphthalen-2-yl-acrylamide (28, Scheme 3)

To a suspension of commercial 3-(2-naphthyl)acrylic acid (0.5 g, 1 eq), EDC.HCl (0.97 g, 2 eq) and DMAP (0.92 g, 3 eq) are added. To resulting mixture, stirred at room temperature for 10 min, Intermediate I (0.627 g, 1 eq) is added and stirring continued overnight at room temperature. The reaction mixture is diluted with DCM (100 ml), washed with 1.5 N HCl (2×50 ml) and water (50 ml), dried and evaporated to dryness to give a yellowish solid (0.95 g, 88% yield). This crude material is purified by flash chromatography using silica gel (230-400 mesh) and 8% MeOH in CFM. LC-ESI-HRMS of [M+H]+ shows 429.0691 Da. Calc. 429.067582 Da, dev. 3.5 ppm.

### 4,5-Dichloro-2-((E)-3-naphthalen-2-yl-acryloylamino)-benzenesulfonic acid (29)

A solution of Intermediate A (0.546 g) in dry DCM (10 ml) is added drop wise to a stirred mixture of 2-amino-4,5-dichloro-benzenesulfonic acid (0.703 g, 1 eq) (prepared as described in DE 4112692) in Py (1 ml) and dry DCM (15 ml). Stirring is continued overnight at room temperature and the mixture is then extracted with 10% sodium hydroxide (2×15 ml). The aqueous layer is acidified with 1.5 N HCl and the precipitate is filtered off and purified by flash chromatography (60-120 mesh silica gel) using 6% MeOH in CFM as eluent, to get the title compound as an off-white solid (270 mg, ˜25% yield). M.p. 228.7-229° C.

### (E)-N-(2-Benzenesulfonylamino-5-chloro-phenyl)-3-naphthalen-2-yl-acrylamide (30)

To a stirred and ice-cooled suspension of Intermediate C (0.25 g) in Py (5 ml), benzenesulfonyl chloride (0.124 g, 1 eq) is added drop wise and the resulting mixture is first allowed to reach room temperature spontaneously and then stirred at room temperature overnight. Evaporation to dryness of the mixture provides a solid residue which is stirred (10 min) first in 1.5 N HCl (10 ml) and then in water (10 ml). The new solid residue is purified by flash chromatography (60-120 mesh silica gel) using CFM as eluent, to give the title compound as a yellow solid (0.05 g, yield ˜15%). M.p. 220-223° C.

### 4-Chloro-2-((E)-3-naphthalen-2-yl-acryloylamino)-benzoylcyanamide (31)

To a stirred solution of Compound 15 (0.25 g) in dry DCM (10 ml), EDC.HCl (0.27 g, 2 eq), DMAP (0.26 g, 2 eq) and cyanamide (0.045 g, 1.5 eq) are added. The brownish solution is stirred at r.t. overnight and is then diluted with DCM (10 ml). This latter solution is washed with 1.5 N HCl (3*15 ml), water (15 ml), dried over MgSO4, and evaporated to dryness. The solid residue (100 mg) is purified by flash chromatography (230-400 mesh silica gel) using 0-7% MeOH in CFM as eluent, to give the title compound as a yellow solid (0.02 g, yield ˜7%). LC-ESI-HRMS of [M+H]+ shows 376,0853 Da. Calc. 376,08528 Da, dev. 0.1 ppm.

### 4-Chloro-2-((E)-3-naphthalen-2-yl-acryloylamino)-benzenesulfonic acid methyl ester (32)

To a stirred and ice-cooled suspension of Compound 19 (0.8 g) in dry DCM (25 ml), methyl trifluoromethanesulfonate (0.37 g, 1.1 eq) and triethylamine (0.31 ml, 1.1 eq) are added, and the resulting mixture is first allowed to reach room temperature spontaneously and then stirred at room temperature overnight. Reaction mixture is diluted with DCM (40 ml), washed with water (50 ml), brine (50 ml), dried over MgSO4 and evaporated to dryness, to give an off-white solid (0.5 mg, 60% mass balance). This crude product is purified by flash column chromatography (on neutral alumina) using 5% AcOEt in HEX as eluent, to give the title compound as an off-white solid (0.1 g, ˜12% yield). M.p. 178.2-180.4° C.

### (E)-N-[5-Chloro-2-(5-oxo-4,5-dihydro-[1,2,4]oxadiazol-3-yl)-phenyl]-3-naphthalen-2-yl-acrylamide (33)

To a suspension of commercial 3-(2-naphthyl)acrylic acid (0.1 g) in dry DCM (5 ml) under nitrogen, triethylamine is added (0.2 ml, 3 eq). To this stirred and ice-cooled solution, EDC.HCl (0.145 g, 1.5 eq) and 1-hydroxybenzotriazole hydrate (0.007 g, 0.1 eq) are added. Stirring and icing is continued for 15 min, then 3-(2-amino-4-chloro-phenyl)-4H-[1,2,4]oxadiazol-5-one (0.125 g, 1 eq) (prepared as described by Valgeirsson et al. in Journal of Medicinal Chemistry 2004 47 (27) 6948-6957) is added, and the reaction mixture is allowed to reach room temperature spontaneously. Stirring at room temperature is continued for 40 hours and the reaction mixture is then diluted with DCM (15 ml), washed with water (15 ml) and brine (15 ml), dried over MgSO4 and evaporated to dryness, to give a yellowish gummy material (0.15 g). This crude material is purified by flash chromatography (230-400 mesh silica gel) using HEX and AcOEt (20%) as eluent, to give 75 mg (38% yield) of the pure title compound. M.p. 230-232° C.

### (E)-3-Naphthalen-2-yl-N-[2-(1H-tetrazol-5-yl)-phenyl]-acrylamide (34)

A solution of Intermediate A (0.273 g) in dry DCM (10 ml) is added drop wise to a stirred mixture of 2-(1H-Tetrazol-5-yl)-phenylamine (0.203 g, 1 eq) (prepared as described by Koguro et al. in Synthesis 1998 6 910-914) in Py (1 ml) and dry DCM (15 ml). Stirring is continued overnight at room temperature and the mixture is then extracted with 10% sodium hydroxide (2×15 ml). The aqueous layer is acidified with 1.5 N HCl and the precipitate is filtered off and purified by crystallisation from a mixture of DMSO and water, to get the title compound as an off-white solid (390 mg, ˜91% yield). M.p. 187-191° C.

### Example 2

### Biological Activity

### Expression and Functional Characterization of the BK Channel

In this example the BK channel opening activity of three cinnamic amide derivatives representative of the invention, i.e. Compound 22 (A), Compound 9 (B) and Compound 19 (C), is determined using BK channels heterologously expressed in Xenopus laevis oocytes.

The electrical current through the BK channel is measured conventional two-electrode voltage clamp. BK current is activated by repeated step protocols. In brief, this protocol goes from a resting membrane potential of −40 mV lasting for 5 seconds to a depolarised step to +30 mV lasting for 1 second. The protocol was repeated continuously.

Having reached a stable current level, Compound A (3 μM), Compound B (1 μM) and Compound C (30 μM) was added. A marked increase in the current activated by depolarisation could be observed.

The results are presented in FIG. 1.

1-21. (canceled)

22. A cinnamic amide derivative of Formula I 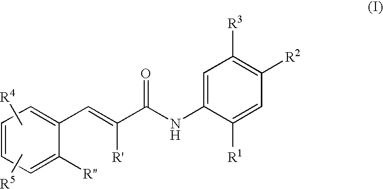
an enantiomer thereof or a mixture of its enantiomers, or a pharmaceutically-acceptable addition salt thereof, wherein
R¹ represents a substituent selected from the group consisting of nitro, amino, hydroxy, carboxy, sulfonic acid, sulfonic acid alkyl ester, sulfamoyl, acetamido, methyl-sulfonyl-amino, phenyl-sulfonyl-amino, N-methyl-sulfonyl-carboxamide (methyl-sulfonyl-amino-carbonyl), N-phenyl-sulfonyl-carboxamide (phenyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl, tetrazolyl-methoxy, 5-oxo-4,5-dihydro-[1,2,4]oxadiazol-3-yl and N-cyano-carboxamide;
R² and R³, independently of each other, represent hydrogen, halo, trifluoromethyl, hydroxy or phenyl, which phenyl may optionally be substituted with halo and/or trifluoromethyl;
R⁴ and R⁵, independently of each other, represent hydrogen, halo, trifluoromethyl, nitro and/or phenyl; or
R⁴ and R⁵ together with the aromatic ring to which they are attached form a benzo-fused carbocyclic aromatic ring; and
R′ and R″ represent hydrogen, or, together with the carbon atoms of the aromatic ring to which they are attached, form a bicyclic carbocyclic or heterocyclic ring selected from indolyl and 2H-chromenyl, which 2H-chromenyl may optionally be substituted with oxo to form a 2-oxo-2H-chromenyl derivative.

23. The cinnamic amide derivative of claim 22, wherein R¹ represents a substituent selected from the group consisting of nitro, amino, hydroxy, carboxy, sulfonic acid, sulfonic acid alkyl ester, sulfamoyl, acetamido, methyl-sulfonyl-amino, phenyl-sulfonyl-amino, N-methyl-sulfonyl-carboxamide (methyl-sulfonyl-amino-carbonyl), N-phenyl-sulfonyl-carboxamide (phenyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl, tetrazolyl-methoxy, 5-oxo-4,5-dihydro-[1,2,4]oxadiazol-3-yl and N-cyano-carboxamide.

24. The cinnamic amide derivative of claim 23, wherein R¹ represents a substituent selected from the group consisting of nitro, amino, sulfamoyl, acetamido, methyl-sulfonyl-amino, phenyl-sulfonyl-amino, N-methyl-sulfonyl-carboxamide (methyl-sulfonyl-amino-carbonyl), N-phenyl-sulfonyl-carboxamide (phenyl-sulfonyl-amino-carbonyl), trifluoromethyl-sulfonyl-amino, trifluoromethyl-acetyl-amino, 2,2,2-trifluoro-1-hydroxy-1-trifluoromethyl-ethyl, tetrazolyl-methoxy, 5-oxo-4,5-dihydro-[1,2,4]oxadiazol-3-yl and N-cyano-carboxamide.

25. The cinnamic amide derivative of claim 22, wherein R² and R³, independently of each other, represent hydrogen, halo, trifluoromethyl, hydroxy or phenyl, which phenyl may optionally be substituted with halo and/or trifluoromethyl.

26. The cinnamic amide derivative of claim 22, wherein
R⁴ and R⁵, independently of each other, represent hydrogen, halo, trifluoromethyl, nitro and/or phenyl; or
R⁴ and R⁵ together with the aromatic ring to which they are attached form a benzo-fused carbocyclic aromatic ring.

27. The cinnamic amide derivative of claim 22, wherein R⁴ and R⁵ together with the aromatic ring to which they are attached form a benzo-fused carbocyclic aromatic ring.

28. The cinnamic amide derivative of claim 22, wherein R′ and R″ represent hydrogen, or, together with the carbon atoms of the aromatic ring to which they are attached, form a bicyclic carbocyclic or heterocyclic ring selected from indolyl and 2H-chromenyl, which 2H-chromenyl may optionally be substituted with oxo to form a 2-oxo-2H-chromenyl derivative.

29. The cinnamic amide derivative of claim 22, wherein
R¹ represents tetrazolyl;
R² represents hydrogen, halo, 4-fluoro-phenyl, 4-chloro-phenyl; and
R³ represents hydrogen or halo.

30. The cinnamic amide derivative of claim 22, wherein
R¹ represents tetrazolyl;
R² represents hydrogen, halo or 4-fluoro-phenyl;
R³ represents hydrogen or halo;
R⁴ represents hydrogen;
R⁵ represents halo; and
R′ and R″, together with the carton atoms to which they are attached and with the aromatic ring, form a bicyclic carbocyclic or heterocyclic ring selected from indolyl and 2H -chromenyl, which 2H-chromenyl may optionally be substituted with oxo to form a 2-oxo-2H-chromenyl derivative.

31. The cinnamic amide derivative of claim 22, wherein
R¹ represents tetrazolyl;
R² represents hydrogen or halo;
R³ represents hydrogen or halo;
R⁴ and R⁵ together with the aromatic ring to which they are attached form a benzo-fused carbocyclic aromatic ring; and
R′ and R″ both represent hydrogen.

32. The cinnamic amide derivative of claim 22 which is
6-Chloro-2H-chromene-3-carboxylic acid [5-chloro-2-(1H-tetrazol-5-yl)-phenyl]-amide;
(E)-N-(5-Chloro-2-hydroxy-phenyl)-3-(3-nitro-phenyl)-acrylamide;
(E)-N-[5-Chloro-2-(1H-tetrazol-5-yl)-phenyl]-3-(4-fluoro-3-trifluoromethyl-phenyl)-acrylamide;
(E)-3-(3,5-Bis-trifluoromethyl-phenyl)-N-(5-chloro-2-hydroxy-phenyl)-acrylamide;
(E)-N-[4-Bromo-2-(2H-tetrazol-5-yl)-phenyl]-3-(3-trifluoromethyl-phenyl)-acrylamide;
5-Chloro-1H-indole-2-carboxylic acid [4′-fluoro-3-(1H-tetrazol-5-yl)-biphenyl-4-yl]-amide;
(E)-3-(3,5-Bis-trifluoromethyl-phenyl)-N-[4′-chloro-3-(2H-tetrazol-5-yl)-biphenyl-4-yl]-acrylamide;
(E)-3-(3,5-Bis-trifluoromethyl-phenyl)-N-[4-iodo-2-(1H-tetrazol-5-yl)-phenyl]-acrylamide;
(E)-3-(3,5-Bis-trifluoromethyl-phenyl)-N-[4-bromo-2-(1H-tetrazol-5-yl)-phenyl]-acrylamide;
(E)-N-[4-Bromo-2-(1H-tetrazol-5-yl)-phenyl]-3-naphthalen-2-yl-acrylamide;
(E)-N-[4-Chloro-2-(1H-tetrazol-5-yl)-phenyl]-3-naphthalen-2-yl-acrylamide;
6-Chloro-2H-chromene-3-carboxylic acid [4-bromo-2-(1H-tetrazol-5-yl)-phenyl]-amide;
5-Chloro-1H-indole-2-carboxylic acid [5-chloro-2-(1H-tetrazol-5-yl)-phenyl]-amide;
(E)-N-[5-Chloro-2-(1H-tetrazol-5-yl)-phenyl]-3-(3,4-dichloro-phenyl)-acrylamide;
4-Chloro-2-((E)-3-naphthalen-2-yl-acryloylamino)-benzoic acid;
(E)-3-Naphthalen-2-yl-N-[2-(2,2,2-trifluoro-1-hydroxy-1-trifluoromethyl-ethyl)-phenyl]-acrylamide;
4-Chloro-2-[(E)-3-(3,4-dichloro-phenyl)-acryloylamino]-benzoic acid;
(E)-N-(2-Acetylamino-5-chloro-phenyl)-3-naphthalen-2-yl-acrylamide;
(E)-N-(5-Chloro-2-nitro-phenyl)-3-naphthalen-2-yl-acrylamide;
4-Chloro-2-((E)-3-naphthalen-2-yl-acryloylamino)-benzenesulfonic acid;
(E)-N-(5-Chloro-2-methanesulfonylamino-phenyl)-3-naphthalen-2-yl-acrylamide;
(E)-N-[5-Chloro-2-(2,2,2-trifluoro-acetylamino)-phenyl]-3-naphthalen-2-yl-acrylamide;
(E)-N-(2-Amino-5-chloro-phenyl)-3-naphthalen-2-yl-acrylamide;
(E)-N-(5-Chloro-4-fluoro-2-sulfamoyl-phenyl)-3-naphthalen-2-yl-acrylamide;
2-((E)-3-Biphenyl-4-yl-acryloylamino)-4-chloro-benzenesulfonic acid;
(E)-N-(5-Chloro-2-trifluoromethanesulfonylamino-phenyl)-3-naphthalen-2-yl-acrylamide;
(E)-N-[5-Chloro-2-(1H-tetrazol-5-ylmethoxy)-phenyl]-3-naphthalen-2-yl-acrylamide;
(E)-N-(2-Benzenesulfonylaminocarbonyl-5-chloro-phenyl)-3-naphthalen-2-yl-acrylamide;
6-Bromo-2-oxo-2H-chromene-3-carboxylic acid [5-chloro-2-(1H-tetrazol-5-yl)-phenyl]-amide;
(E)-N-(5-Chloro-2-methanesulfonylaminocarbonyl-phenyl)-3-naphthalen-2-yl-acrylamide;
4,5-Dichloro-2-((E)-3-naphthalen-2-yl-acryloylamino)-benzenesulfonic acid;
(E)-N-(2-Benzenesulfonylamino-5-chloro-phenyl)-3-naphthalen-2-yl-acrylamide;
4-Chloro-2-((E)-3-naphthalen-2-yl-acryloylamino)-benzoylcyanamide;
4-Chloro-2-((E)-3-naphthalen-2-yl-acryloylamino)-benzenesulfonic acid methyl ester;
(E)-N-[5-Chloro-2-(5-oxo-4,5-dihydro-[1,2,4]oxadiazol-3-yl)-phenyl]-3-naphthalen-2-yl-acrylamide; or
(E)-3-Naphthalen-2-yl-N-[2-(1H-tetrazol-5-yl)-phenyl]-acrylamide;
or a pharmaceutically-acceptable addition salt thereof.

33. A pharmaceutical composition comprising a therapeutically effective amount of the cinnamic amide derivative of claim 22, or a pharmaceutically-acceptable addition salt thereof, or a prodrug thereof, together with one or more adjuvants, excipients, carriers and/or diluents.

34. A kit of parts comprising at least two separate unit dosage foams (A) and (B1) or (B2):
(A) a cinnamic amide derivative according to claim 22; and
(B1) a phosphodiesterase inhibitor; or
(B2) an agent that potentiates endothelium-derived hyperpolarizing factor-mediated responses; and optionally
(C) instructions for the simultaneous, sequential or separate administration of the cinnamic amide derivative of A, and the phosphodiesterase inhibitor of B1, or an agent that potentiates endothelium-derived hyperpolarizing factor-mediated responses of B2, to a patient in need thereof.

35. A method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the cinnamic amide derivative according to claim 22.

36. A method of treatment or alleviation of a sexual dysfunction, which method comprises the step of administering to such a living animal body in need thereof, a therapeutically effective amount of a combination of
(A) a cinnamic amide derivative according to claim 22; and
(B1) a phosphodiesterase inhibitor; or
(B2) an agent that potentiates endothelium-derived hyperpolarizing factor-mediated responses;
or pharmaceutically-acceptable addition salts thereof.

37. The method according to claim 35, wherein the disease, disorder or condition is a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhoea, narcolepsy, sleeping disorders, sleep apnoea, Reynaud's disease, intermittent claudication, Sjögren's syndrome, xerostomia, arrhythmia, cardiovascular disorders, hypertension, myotonic dystrophy, myotonic muscle dystrophia, spasticity, xerostomia, diabetes Type II, hyperinsulinemia, premature labour, cancer, brain tumours, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma), baldness, cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy or cardiomyopathy.

38. The method of claim 36, wherein the sexual dysfunction is a male sexual dysfunction, a female sexual dysfunction or a male erectile dysfunction.

39. The method according to claim 36, wherein the phosphodiesterase inhibitor of is sildenafil, tadalafil or vardenafil; and the agent that potentiates endothelium-derived hyperpolarizing factor-mediated responses is calcium dobesilate.
